(19) United States
(12) Patent Application Publication
(10) Pub. No.: US 2007/0287738 A1
(43) Pub. Date: Dec. 13, 2007

(54) Substituted Cyanopyridines as protein kinase inhibitors

(76) Inventors: Derek Cecil Cole, New City, NY (US); Magda Asselin, Mahwah, NJ (US); Diane Harris Boschelli, New City, NY (US); Allan Wissner, Ardsley, NY (US); Yanong Daniel Wang, Warren, NJ (US); Amarnauth Shastrie Prashad, New City, NY (US); Russell Dushin, Garrison, NY (US)

Correspondence Address: KIRKPATRICK & LOCKHART PRESTON GATES ELLIS LLP;(FORMERLY KIRKPATRICK&LOCKHART NICHOLSON GRAHAM), STATE STREET FINANCIAL CENTER, ONE LINCOLN STREET, BOSTON, MA, 02111-2950, US

(21) Appl. No.: 11/818,159

(22) Filed: Jun. 13, 2007

Related U.S. Application Data
(60) Provisional application No. 60/813,182, filed on Jun. 13, 2006.

(51) Int. Cl. A61K 31/44; C07D 213/84

(52) U.S. Cl. 514/344; 546/286

(57) ABSTRACT

The present teachings provide compounds of formula I

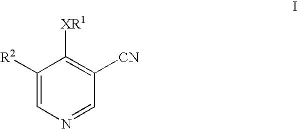

and their pharmaceutically acceptable salts, hydrates, and esters, wherein R¹, R², and X are as defined herein. The present teachings also provide methods of making the compounds of formula I, and methods of treating autoimmune and inflammatory diseases by administering a therapeutically effective amount of a compound or compounds of formula I to a mammal including a human.

### CROSS REFERENCE TO RELATED APPLICATIONS

This application claims priority to and the benefit of U.S. Provisional Patent Application Ser. No. 60/813,182, filed on Jun. 13, 2006, the disclosure of which is incorporated by reference herein in its entirety.

### FIELD

The present teachings relate to substituted 3-cyanopyridines (also known as nicotinonitriles) that are capable of inhibiting protein kinases. The present teachings also relate to methods for the preparation of the substituted cyanopyridines and methods of their use. For example, the compounds of the present teachings can be useful for the treatment of autoimmune and inflammatory diseases such as asthma and arthritis.

### INTRODUCTION

Protein kinases are enzymes that catalyze the transfer of a phosphate group from adenosine triphosphate (ATP) to an amino acid residue (e.g., tyrosine, serine, threonine or histidine) on a protein. Regulation of these protein kinases is essential for the control of a wide variety of cellular events including proliferation and migration. A large number of diseases including various inflammatory diseases and autoimmune diseases such as asthma, colitis, multiple sclerosis, psoriasis, arthritis, rheumatoid arthritis, osteoarthritis, and joint inflammation, are associated with abnormal cellular events that are mediated by these kinases. See, e.g., Salek-Ardakami, S. et al. (2004), J. Immunology, 173(10): 6440-47; Marsland, B. et al. (2004), J. Exp. Med, 200(2): 181-89; Tan, S, et al. (2006), J. Immunology, 176: 2872-79; Salek-Ardakami, S. et al. (2005), J. Immunology, 175(11): 7635-41; Anderson, K. et al. (2006), Autoimmunity, 39(6): 469-78; Healy, A. et al. (2006), J. Immunology, 177(3): 1886-93; Sun, Z. et al. (2000), Nature, 404: 402-7; and Pfeifhofer, C. et al. (2003), J. Exp. Med., 197(11): 1525-35.

One class of serine/threonine kinases is the protein kinase C (PKC) family. This group of kinases consists of 10 members that share sequence and structural homology. The PKCs are divided into 3 groups and include the classic, the novel, and the atypical isoforms. The theta isoform (PKCθ) is a member of the novel calcium-independent class of PKCs (Baier, G. et al. (1993), J. Biol. Chem., 268: 4997-5004). PKCθ is highly expressed in T cells (Mischak, H. et al. (1993), FEBS Lett., 326: 51-5), with some expression reported in mast cells (Liu, Y. et al. (2001), J. Leukoc. Biol., 69: 831-40), endothelial cells (Mattila, P. et al. (1994), Life Sci., 55: 1253-60), and skeletal muscles (Baier, G. et al. (1994), Eur. J. Biochem., 225: 195-203). It has been shown that PKCθ plays an essential role in T cell receptor (TCR)-mediated signaling (Tan, S. L. et al. (2003), Biochem. J, 376: 545-52). Specifically, it has been observed that inhibiting PKCθ signal transduction, as demonstrated with two independent PKCθ knockout mouse lines, will result in defects in T cell activation and interleukin-2 (IL-2) production (Sun, Z. et al. (2000), Nature, 404: 402-7; Pfeifhofer, C. et al. (2003), J. Exp. Med, 197: 1525-35). It also has been shown that PKCθ-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al. (2004), J. Exp. Med., 199: 743-52; Marsland, B. J. et al. (2004), J. Exp. Med., 200: 181-9). The impaired Th2 cell responses result in reduced levels of interleukin-4 (IL-4) and immunoglobulin E (IgE), contributing to the AHR and inflammatory pathophysiology.

Evidence also exists that PKCθ participates in the IgE receptor (FceRI)-mediated response of mast cells (Liu, Y. et al. (2001), J. Leukoc. Biol., 69: 831-840). In human-cultured mast cells (HCMC), it has been demonstrated that PKC kinase activity rapidly localizes (in less than five minutes) to the membrane following FceRI cross-linking (Kimata, M. et al. (1999), Biochem. Biophys. Res. Commun., 257(3): 895-900). A recent study examining in vitro activation of bone marrow mast cells (BMMCs) derived from wild-type and PKCθ-deficient mice shows that upon FceRI cross-linking, BMMCs from PKCθ-deficient mice produced reduced levels of interleukin-6 (IL-6), tumor necrosis factor-alpha (TNFα), and interleukin-13 (IL-13) in comparison with BMMCs from wild-type mice, suggesting a potential role for PKCθ in mast cell cytokine production in addition to T cell activation (Ciarletta, A. B. et al. (2005), poster presentation at the 2005 American Thorasic Society International Conference).

Other serine/threonine kinases include those of the mitogen-activated protein kinase (MAPK) pathway which consists of the MAP kinases (MAPK) (e.g., erk) and the MAPK kinases (MAPKK) (e.g., mek and their substrates). Members of the raf family of kinases phosphorylate residues on mek. The cyclin-dependent kinases (cdks), including cdc2/cyclin B, cdk2/cyclin A, cdk2/cyclin E and cdk4/cyclin D, and others, are serine/threonine kinases that regulate mammalian cell division. Additional serine/threonine kinases include the protein kinases A and B. These kinases, known as PKA or cyclic AMP-dependent protein kinase and PKB (Akt), play key roles in signal transduction pathways.

Tyrosine kinases (TKs) are divided into two classes: the non-transmembrane TKs and transmembrane growth factor receptor TKs (RTKs). Growth factors, such as epidermal growth factor (EGF), bind to the extracellular domain of their partner RTK on the cell surface which activates the RTK, initiating a signal transduction cascade that controls a wide variety of cellular responses. In addition to EGF, there are several other RTKs including FGFR (the receptor for fibroblast growth factor (FGF)); flk-1 (also known as KDR), and flt-1 (the receptors for vascular endothelial growth factor (VEGF)); and PDGFR (the receptor for platelet derived growth factor (PDGF)). Other RTKs include tie-1 and tie-2, colony stimulating factor receptor, the nerve growth factor receptor, and the insulin-like growth factor receptor. In addition to the RTKs there is another family of TKs termed the cytoplasmic protein or non-receptor TKs. The cytoplasmic protein TKs have intrinsic kinase activity, are present in the cytoplasm and nucleus, and participate in diverse signaling pathways. There are a large number of non-receptor TKs including Abl, Jak, Fak, Syk, Zap-70 and Csk, and the Src family of kinases (SFKs) which include Src, Lck, Lyn, Fyn and others.

Certain pyridine and pyrimidine derivatives have been noted as kinase inhibitors. These compounds differ both in nature and placement of substituents at various positions when compared to the compounds of the present teachings.

### SUMMARY

The present teachings relate to substituted 3-cyanopyridines of formula I:

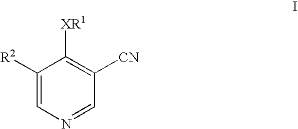

and their pharmaceutically acceptable salts, hydrates, and esters, wherein R¹, R², and X are defined as described herein.

The present teachings also relate to pharmaceutical compositions that include a pharmaceutically effective amount of one or more compounds of formula I (including their pharmaceutically acceptable salts, hydrates, and esters) and a pharmaceutically acceptable carrier or excipient. Another aspect of the present teachings relates to methods of preparing the compounds of formula I and their pharmaceutically acceptable salts, hydrates, and esters. The present teachings also provide methods of using the compounds of formula I and their pharmaceutically acceptable salts, hydrates, and esters. In some embodiments, the present teachings provide methods of treating autoimmune and inflammatory diseases, such as asthma, colitis, multiple sclerosis, psoriasis, arthritis, rheumatoid arthritis, osteoarthritis, and joint inflammation, which include administering a therapeutically effective amount of one or more compounds of formula I (or their pharmaceutically acceptable salts, hydrates, or esters) to a mammal including a human.

### DETAILED DESCRIPTION

The present teachings provide compounds of formula I:

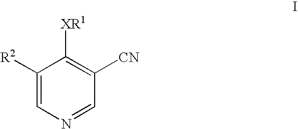

and their pharmaceutically acceptable salts, hydrates, and esters, wherein:
X is selected from a) —NR³—Y—, b) —O—Y—, c) —S(O)ₘ—Y—, d) —S(O)ₘNR³—Y—, e) —NR³S(O)ₘ—Y—, f) —C(O)NR³—Y—, g) —C(S)NR³—Y—, h) —NR³C(O)—Y—, i) —NR³C(S)Y—, j) —C(O)O—Y—, k) —OC(O)—Y—, and l) a covalent bond;
Y, at each occurrence, independently is selected from a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, and e) a covalent bond;
R¹ is a phenyl group optionally substituted with 1-4 —Y—R⁴ groups;
R² is a C₆₋₁₄ aryl group or a 5-14 membered heteroaryl group, wherein each group optionally is substituted with 1-4 groups independently selected from —Y—R⁴ and —O—Y—R⁴;
R³ is selected from a) H, b) a C₁₋₁₀ alkyl group, c) a C₂₋₁₀ alkenyl group, d) a C₂₋₁₀ alkynyl group, and e) a C₁₋₁₀ haloalkyl group;
R⁴, at each occurrence, independently is selected from a) halogen, b) —CN, c) —NO₂, d) oxo, e) —O—Y—R⁵, f) —NR⁶—Y—R⁷, g) —N(O)R⁶—Y—R⁷, h) —S(O)ₘ—Y—R⁵, i) —S(O)ₘO—Y—R⁵, j) —S(O)ₘNR⁶—Y—R⁷, k) —C(O)—Y—R⁵, l) —C(O)O—Y—R⁵, m) —C(O)NR⁶—Y—R⁷, n) —C(S)NR⁶—Y—R⁷, o) a C₁₋₁₀ alkyl group, p) a C₂₋₁₀ alkenyl group, q) a C₂₋₁₀ alkynyl group, r) a C₋₁₀ haloalkyl group, s) a C₃₋₁₄ cycloalkyl group, t) a C₆₋₁₄ aryl group, u) a 3-14 membered cycloheteroalkyl group, and v) a 5-14 membered heteroaryl group, wherein each of o)-v) optionally is substituted with 1-4 —Y—R⁸ groups;
R⁵, at each occurrence, independently is selected from a) H, b) —C(O)R⁹, c) —C(O)OR⁹, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₄ cycloalkyl group, i) a C₆₋₁₄ aryl group, j) a 3-14 membered cycloheteroalkyl group, and k) a 5-14 membered heteroaryl group, wherein each of d)-k) optionally is substituted with 1-4 —Y—R⁸ groups;
R⁶ and R⁷, at each occurrence, independently are selected from a) H, b) —O—Y—R⁹, c) —S(O)ₘ—Y—R⁹, d) —S(O)ₘO—Y—R⁹, e) —C(O)—Y—R⁹, f) —C(O)O—Y—R⁹, g) —C(O)NR¹⁰—Y—R¹¹, h) —C(S)NR¹⁰—Y—R¹¹, i) a C₁₋₁₀ alkyl group, j) a C₂₋₁₀ alkenyl group, k) a C₂₋₁₀ alkynyl group, 1) a C₁₋₁₀ haloalkyl group, m) a C₃₋₁₄ cycloalkyl group, n) a C₆₋₁₄ aryl group, o) a 3-14 membered cycloheteroalkyl group, and p) a 5-14 membered heteroaryl group, wherein each of i)-p) optionally is substituted with 1-4 —Y—R⁸ groups;
R⁸, at each occurrence, independently is selected from a) halogen, b) —CN, c) —NO₂, d) oxo, e) —O—Y—R⁹, f) —NR¹⁰—Y—R¹¹, g) —N(O)R¹⁰—Y—R¹¹, h) —S(O)ₘ—Y—R⁹, i) —S(O)ₘO—Y—R⁹, j) —S(O)ₘNR¹⁰—Y—R¹¹, k) —C(O)—Y—R⁹, l)—C(O)O—Y—R⁹, m) —C(O)NR¹⁰—Y—R¹¹, n) —C(S)NR¹⁰—Y—R¹¹, o) a C₁₋₁₀ alkyl group, p) a C₂₋₁₀ alkenyl group, q) a C₂₋₁₀ alkynyl group, r) a C₁₋₁₀ haloalkyl group, s) a C₃₋₁₄ cycloalkyl group, t) a C₆₋₁₄ aryl group, u) a 3-14 membered cycloheteroalkyl group, and v) a 5-14 membered heteroaryl group, wherein each of o)-v) optionally is substituted with 1-4 —Y—R¹² groups;
R⁹, at each occurrence, independently is selected from a) H, b) —C(O)—C₁₋₁₀ alkyl, c) —C(O)OH, d) —C(O)O—C₁₋₁₀ alkyl, e) a C₁₋₁₀ alkyl group, f) a C₂₋₁₀ alkenyl group, g) a C₂₋₁₀ alkynyl group, h) a C₁₋₁₀ haloalkyl group, i) a C₃₋₁₄ cycloalkyl group, j) a C₆₋₁₄ aryl group, k) a 3-14 membered cycloheteroalkyl group, and l) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Y—R¹² groups;
R¹⁰ and R¹¹, at each occurrence, independently are selected from a) H, b) —OH, c) —SH, d) —NH₂, e) —NH—C₁₋₁₀ alkyl, f) —N(C₁₋₁₀ alkyl)₂, g) —S(O)ₘ—C₁₋₁₀ alkyl, h) —S(O)₂OH, i) —S(O)ₘ—OC₁₋₁₀ alkyl, j) —C(O)C₁₋₁₀ alkyl, k) —C(O)OH, l) —C(O)—OC₁₋₁₀ alkyl, m) —C(O)NH₂, n) —C(O)NH-C₁₋₁₀ alkyl, o) —C(O)N(C₁₋₁₀ alkyl)₂, p) —C(S)NH₂, q) —C(S)NH—C₁₋₁₀ alkyl, r) —C(S)N(C₁₋₁₀ alkyl)₂, s) a C₁₋₁₀ alkyl group, t) a C₂₋₁₀ alkenyl group, u) a C₂₋₁₀ alkynyl group, v) a C₁₋₁₀ alkoxy group, w) a C₁₋₁₀ haloalkyl group, x) a C₃₋₁₄ cycloalkyl group, y) a C₆₋₁₄ aryl group, z) a 3-14 membered cycloheteroalkyl group, and aa) a 5-14 membered heteroaryl group, wherein each of the C₁₋₁₀ alkyl group, the C₂₋₁₀ alkenyl group, the C₂₋₁₀ alkynyl group, the C₁₋₁₀ alkoxy group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Y—R¹² groups;
R¹², at each occurrence, independently is selected from a) halogen, b) —CN, c) —NO₂, d) oxo, e) —OH, f) —NH₂, g) —NH(C₁₋₁₀ alkyl), h) —N(C₁₋₁₀ alkyl)₂, i) —SH, j) —S(O)ₘ—C₁₋₁₀ alkyl, k) —S(O)₂OH, l)—S(O)ₘ—OC₁₋₁₀ alkyl, m) —C(O)—C₁₋₁₀ alkyl, n) —C(O)OH, o) —C(O)—OC₁₋₁₀ alkyl, p) —C(O)NH₂, q) —C(O)NH—C₁₋₁₀ alkyl, r) —C(O)N(C₁₋₁₀ alkyl)₂, s) —C(S)NH₂, t) —C(S)NH—C₁₋₁₀ alkyl, u) —C(S)N(C₁₋₁₀ alkyl)₂, v) a C₁₋₁₀ alkyl group, w) a C₂₋₁₀ alkenyl group, x) a C₂₋₁₀ alkynyl group, y) a C₁₋₁₀ alkoxy group, z) a C₁₋₁₀ haloalkyl group, aa) a C₃₋₁₄ cycloalkyl group, ab) a C₆₋₁₄ aryl group, ac) a 3-14 membered cycloheteroalkyl group, and ad) a 5-14 membered heteroaryl group; and
m is 0, 1, or 2.

In some embodiments, the pyridine ring can be oxidized on the nitrogen atom to provide the corresponding N-oxide having the formula I′:

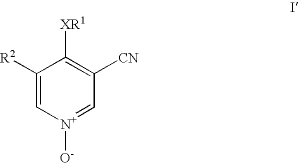

wherein R¹, R², and X are as defined herein.

In some embodiments, X can be selected from —NR³—Y—, —O—Y—, and a covalent bond. For example, X can be selected from —NH—, —N(CH₃), —NH—CH₂—, —NH—CH₂CH₂—, —NH—CH₂CH₂CH₂—, —O—, and a covalent bond. In particular embodiments, X can be —NH—.

In certain embodiments, R¹ can be selected from:

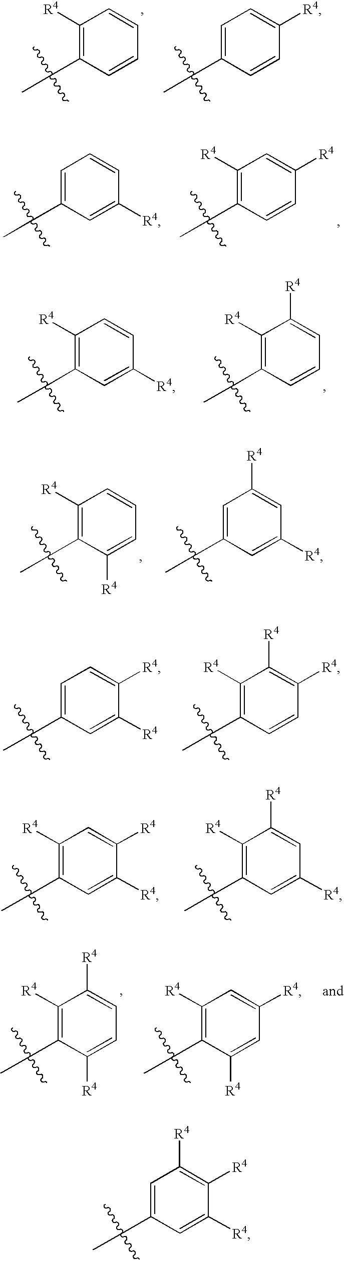

wherein R⁴ is as defined herein. In particular embodiments, R⁴, at each occurrence, can be independently selected from —F, —Cl, —Br, —CN, —NO₂, —O—Y—R⁵, —C(O)Y—R⁵, —C(O)O—Y—R⁵, —NR⁶—Y—R⁷, and a C₁₋₆ alkyl group. For example, R⁴, at each occurrence, can be independently selected from —F, —Cl, —Br, —O—C₁₋₃ alkyl, —O-phenyl, and a C₁₋₃ alkyl group.

In some embodiments, R² can be selected from a phenyl group, a C₈₋₁₄ aryl group, and a 5-14 membered heteroaryl group, wherein each of these groups can be optionally substituted with 1-4 groups independently selected from —Y—R⁴ and —O—Y—R⁴, wherein Y and R⁴ are as defined herein. For example, R² can be selected from a phenyl group, a pyridyl group, a pyrimidyl group, a pyrazinyl group, a furyl group, a thienyl group, a thiazolyl group, an oxazolyl group, a benzofuranyl group, a benzothienyl group, an indolyl group, a benzodioxinyl group, a benzodioxolyl group, a benzodioxanyl group, a dibenzofuranyl group, a dibenzothienyl group, a benzoindolyl group, an indanyl group, an indenyl group, an isothiazolyl group, a pyridazinyl group, a pyrazolyl group, a tetrahydronaphthyl group, an isoxazolyl group, a quinolinyl group, a naphthyl group, an imidazolyl group, and a pyrrolyl group, wherein each of these groups can be optionally substituted with 1-4 groups independently selected from —Y—R⁴ or —O—Y—R⁴, wherein Y and R⁴ are as defined herein.

In certain embodiments, R² can be

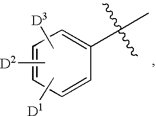

wherein D¹, D², and D³ independently can be H, a —Y—R⁴ group, or an —O—Y—R⁴ group, wherein Y and R⁴ are as defined herein.

For example, at least one of D¹, D², and D³ can be a —Y—R⁴ group or an —O—Y—R⁴ group, wherein Y, at each occurrence, can be independently a divalent C₁₋₄ alkyl group or a covalent bond, and R⁴, at each occurrence, can be independently selected from a halogen, —CN, —NO₂, —O—Y—R⁵, —NR⁶—Y—R⁷, —S(O)₂—Y—R⁵, —S(O)₂NR⁶—Y—R⁷, —C(O)—Y—R⁵, —C(O)O—Y—R⁵, —C(O)NR⁶—Y—R⁷, a C₁₋₁₀ alkyl group, a C₁₋₁₀ haloalkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R⁸ groups, wherein Y, R⁵, R⁶, R⁷, and R⁸ are as defined herein.

In certain embodiments, at least one of D¹, D², and D³ can be an —O—(CH₂)ₙ—R⁴ group, wherein n, at each occurrence, independently can be 0, 1, 2, 3, or 4, and R⁴, at each occurrence, can be independently selected from F, Cl, Br, —NO₂, —O—Y—R⁵, —NR⁶—Y—R⁷, S(O)₂—Y—R⁵, —S(O)₂NR⁶—Y—R⁷, —C(O)NR⁶—Y—R⁷, a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R⁸ groups, wherein Y, R⁵, R⁶, R⁷, and R⁸ are as defined herein. In particular embodiments, at least one of D¹, D², and D3 can be —O—(CH₂)ₙNR⁶—Y—R⁷ or an —O—(CH₂)ₙ-3-14 membered cycloheteroalkyl group, wherein the 3-14 membered cycloheteroalkyl group can be optionally substituted with 1-4 —Y—R⁸ groups, wherein Y, R⁶, R⁷, and R⁸ are as defined herein, and n, at each occurrence, independently can be 0, 1, 2, 3, or 4.

In some embodiments, at least one of D¹, D², and D³ can be —(CH₂)ₙNR⁶—Y—R⁷ or a —(CH₂)ₙ-3-14 membered cycloheteroalkyl group, wherein the 3-14 membered cycloheteroalkyl group can be optionally substituted with 1-4 —Y—R⁸ groups, Y, R⁶, R⁷, and R⁸ are as defined herein, and n, at each occurrence, independently can be 0, 1, 2, 3, or 4.

In embodiments where at least one of D¹, D², and D³ can be an —O—(CH₂)ₙNR⁶—Y—R⁷ group or a —(CH₂)ₙNR⁶—Y—R⁷ group, the —O—(CH₂)ₙNR⁶—Y—R⁷ group and the —(CH₂)ₙNR⁶—Y—R⁷ group can be —O—(CH₂)ₙNH—Y—R⁷ or —O—(CH₂)ₙN(CH₃)—Y—R⁷, and —(CH₂)ₙNH—Y—R⁷ or —(CH₂)ₙN(CH₃)—Y—R⁷, respectively, wherein Y, at each occurrence, can be independently a divalent C₁₋₄ alkyl group or a covalent bond, and R⁷, at each occurrence, can be independently selected from —O—Y—R⁹, —C(O)Y—R⁹, —C(O)O—Y—R⁹, —C(O)NR¹⁰—Y—R¹¹, a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R¹² groups, wherein Y and R¹² are as defined herein. For example, the C₃₋₁₄ cycloalkyl group, the C₆₋₁₄ aryl group, the 3-14 membered cycloheteroalkyl group, and the 5-14 membered heteroaryl group can be selected from a cyclopentyl group, a cyclohexyl group, a phenyl group, a pyrrolidinyl group, a morpholinyl group, a piperazinyl group, a piperidinyl group, an azepanyl group, a diazepanyl group, a thiomorpholinyl group, a furyl group, an imidazolyl group, and a pyridinyl group, wherein each of these groups can be optionally substituted with 1-4 —Y—R¹² groups, wherein Y and R¹² are as defined herein.

In embodiments where at least one of D¹, D², and D³ can be an —O—(CH₂)ₙ-3-14 membered cycloheteroalkyl group or a —(CH₂)ₙ-3-14 membered cycloheteroalkyl group, the 3-14 membered cycloheteroalkyl group can be selected from a pyrrolidinyl group, a morpholinyl group, a piperazinyl group, a piperidinyl group, an azepanyl group, a diazepanyl group, and a thiomorpholinyl group, wherein each of these groups can be optionally substituted with 1-4 —Y—R⁸ groups, wherein Y and R⁸ are as defined herein. For example, Y, at each occurrence, can be independently a divalent C₁₋₄ alkyl group or a covalent bond, and R⁸, at each occurrence, can be independently an oxo group, —O—Y—R⁹, —NR¹⁰—Y—R¹¹, —S(O)ₙ—Y—R⁹, —C(O)O—Y—R⁹, a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R¹² groups, wherein Y and R¹² are as defined herein. For example, the C₃₋₁₄ cycloalkyl group, the C₆₋₁₄ aryl group, the 3-14 membered cycloheteroalkyl group, and the 5-14 membered heteroaryl group can be selected from a cyclopentyl group, a cyclohexyl group, a phenyl group, a pyrrolidinyl group, a morpholinyl group, a piperazinyl group, a piperidinyl group, an azepanyl group, a diazepanyl group, a thiomorpholinyl group, a furyl group, an imidazolyl group, and a pyridinyl group, wherein each of these groups can be optionally substituted with 1-4 —Y—R¹² groups, wherein Y and R¹² are as defined herein.

Alternatively or concurrently, at least one of D¹, D², and D³ can be selected from halogen, —CN, —NO₂, —S(O)₂—Y—R⁵, —S(O)₂NR⁶—Y—R⁷, —C(O)O—Y—R⁵, —C(O)NR⁶—Y—R⁷, a C₁₋₁₀ alkyl group, and a C₁₋₁₀ haloalkyl group, wherein Y, R⁵, R⁶, and R⁷ are as defined herein.

In some embodiments, at least two of D¹, D², and D³ can be —O—(CH₂)ₙ—R⁴ groups, wherein n, at each occurrence, independently can be 0, 1, 2, 3, or 4, and R⁴, at each occurrence, can be independently selected from F, Cl, Br, —NO₂, —O—Y—R⁵, —NR⁶—Y—R⁷, —S(O)₂—Y—R⁵, —S(O)₂NR⁶—Y—R⁷, —C(O)NR⁶—Y—R⁷, a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R⁸ groups, wherein Y, R⁵, R⁶, R⁷, and R⁸ are as defined herein.

In certain embodiments, at least two of D¹, D², and D³ can be independently an —O—CH₃ group or an —O—(CH₂)ₙ—O—Y—R⁵ group, wherein Y and R⁵ are as defined herein, and n, at each occurrence, independently can be 0, 1, 2, 3, or 4. In certain embodiments, two of D¹, D², and D³ can be —O—CH₃ groups. In other embodiments, two of D¹, D², and D³ can be —O—(CH₂)ₙ—O—Y—R⁵ groups or alternatively, an —O—CH₃ group and an —O—(CH₂)ₙ—O—Y—R⁵ group, wherein Y and R⁵ are as defined herein, and n, at each occurrence, independently can be 0, 1, 2, 3, or 4.

In certain embodiments, at least one of D¹, D², and D³ can be —O—CH₃, and at least one of D¹, D², and D³ can be an —O—(CH₂)ₙNR⁶—Y—R⁷ group or an —O—(CH₂)ₙ-3-14 membered cycloheteroalkyl group, wherein the 3-14 membered cycloheteroalkyl group can be optionally substituted with 1-4 —Y—R⁸ groups, wherein Y, R⁶, R⁷, and R⁸ are as defined herein, and n, at each occurrence, independently can be 0, 1, 2, 3, or 4.

In some embodiments, one of D¹, D², and D³ can be

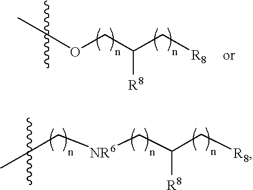

wherein R⁸, at each occurrence, independently can be selected from —O—Y—R⁹, —NR¹⁰—Y—R¹¹, a C₆₋₁₄ aryl group, and a 5-14 membered heteroaryl group, wherein each of the C₆₋₁₄ aryl group and the 5-14 membered heteroaryl group can be optionally substituted with 1-4 —Y—R¹² groups, wherein Y, R⁹, R¹⁰, R¹¹, and R¹² are as defined herein, and n, at each occurrence, independently can be 0, 1, 2, 3, or 4.

In certain embodiments, at least one of D¹, D², and D³ can be a C₆₋₁₄ aryl group or a 5-14 membered heteroaryl group, wherein each of these groups can be optionally substituted with 1-4 —Y—R⁸ groups, wherein Y and R⁸ are as defined herein. For example, at least one of D¹, D², and D³ can be selected from a benzothienyl group, a benzofuryl group, a furyl group, a pyridyl group, a pyrimidinyl group, a pyrrolyl group, and a thienyl group, wherein each of these groups can be optionally substituted with 1-4 —Y—R⁸ groups, wherein Y and R⁸ are as defined herein. In particular embodiments, Y, at each occurrence, can be independently a C₁₋₄ alkyl group or a covalent bond, and R⁸ can be independently selected from a halogen, —CN, —NO₂, —O—Y—R⁹, —NR¹⁰—Y—R¹¹, —C(O)—Y—R⁹, —C(O)NR¹⁰—Y—R¹¹, —S(O)₂—Y—R⁹, —S(O)₂NR¹⁰—Y—R¹¹, and a 3-14 membered cycloheteroalkyl group optionally substituted with a C₁₋₄ alkyl group, wherein Y, R⁹, R¹⁰, and R¹¹ are as defined herein.

In other embodiments, R² can be a C₈₋₁₄ bicyclic aryl group or a 5-14 membered heteroaryl group, where each of these groups can be optionally substituted with 1-4 groups independently selected from —Y—R⁴ groups and —O—Y—R⁴ groups, wherein Y and R⁴ are as defined herein.

In particular embodiments, R² can be selected from a benzothienyl group, a benzofuryl group, a furyl group, a pyridyl group, a pyrimidinyl group, a pyrazinyl group, a thienyl group, an imidazolyl group, an isoxazolyl group, a thiazolyl group, an oxazolyl group, an indolyl group, a benzodioxolyl group, a benzodioxanyl group, and a dibenzofuranyl group, wherein each of these groups can be optionally substituted with 1-4 groups independently selected from a —(CH₂)ₙ—R⁴ group and an —O—(CH₂)ₙ—R⁴ group, wherein n, at each occurrence, independently can be 0, 1, 2, 3, or 4, and R⁴, at each occurrence, can be independently —NR⁶—Y—R⁷ or a 3-14 membered cycloheteroalkyl group optionally substituted with 1-4-Y—R⁸ group, wherein Y, R⁶, R⁷ and R⁸ are as defined herein.

For example, R⁴ can be —O—(CH₂)ₙNH—Y—R⁷, —O—(CH₂)ₙN(CH₃)—Y-R⁷, —(CH₂)ₙNH—Y—R⁷, or —(CH₂)ₙN(CH₃)—Y—R⁷, wherein Y, at each occurrence, can be independently a divalent C₁₋₄ alkyl group or a covalent bond, and R⁷, at each occurrence, can be independently selected from —O—Y—R⁹, —C(O)—Y—R⁹, —C(O)O—Y—R⁹, —C(O)NR¹⁰—Y—R¹¹, a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R¹² groups, wherein Y and R¹² are as defined herein. In particular embodiments, R⁷ can be a C₃₋₁₄ cycloalkyl group, a C₆₋₁₄ aryl group, a 3-14 membered cycloheteroalkyl group, or a 5-14 membered heteroaryl group selected from a cyclopentyl group, a cyclohexyl group, a phenyl group, a pyrrolidinyl group, a morpholinyl group, a piperazinyl group, a piperidinyl group, an azepanyl group, a diazepanyl group, a thiomorpholinyl group, a furyl group, an imidazolyl group, and a pyridinyl group, wherein each of these groups can be optionally substituted with 1-4 —Y—R¹² groups, wherein Y and R¹² are as defined herein.

Alternatively, R⁴ can be an —O—(CH₂)ₙ-3-14 membered cycloheteroalkyl group or a a —(CH₂)ₙ-3-14 membered cycloheteroalkyl group, wherein the 3-14 membered cycloheteroalkyl group can be selected from a pyrrolidinyl group, a morpholinyl group, a piperazinyl group, a piperidinyl group, an azepanyl group, a diazepanyl group, and a thiomorpholinyl group, wherein each of these groups can be optionally substituted with 1-4 —Y—R⁸ groups, wherein Y and R⁸ are as defined herein. For example, Y, at each occurrence, can be independently a divalent C₁₋₄ alkyl group or a covalent bond, and R⁸, at each occurrence, can be independently an oxo group, —O—Y—R⁹, —NR¹⁰—Y—R¹¹, —S(O)ₘ—Y—R⁹, —C(O)O—Y—R⁹, a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group can be optionally substituted with 1-4 —Y—R¹² groups, wherein Y and R¹² are as defined herein. For example, R⁸ can be a C₃₋₁₄ cycloalkyl group, a C₆₋₁₄ aryl group, a 3-14 membered cycloheteroalkyl group, and a 5-14 membered heteroaryl group selected from a cyclopentyl group, a cyclohexyl group, a phenyl group, a pyrrolidinyl group, a morpholinyl group, a piperazinyl group, a piperidinyl group, an azepanyl group, a diazepanyl group, a thiomorpholinyl group, a furyl group, an imidazolyl group, and a pyridinyl group, wherein each of these groups can be optionally substituted with 1-4 —Y—R¹² groups, wherein Y and R¹² are as defined herein.

It should be understood that the present teachings can exclude certain embodiments of compounds within the genus of compounds identified by formula I. For example, when R¹ is a 3-chloro-4-fluorophenyl group, the present teachings can exclude compounds where R² is a 2-[(1H-imidazol-5-ylmethyl)amino]phenyl group.

Compounds of the present teachings include the compounds presented in Table 1 below.

TABLE 1

| Compound | Compound Name |
|---|---|
| 101 | 4-[(3-chlorophenyl)amino]-5-(3,4-dimethoxyphenyl)nicotinonitrile |
| 102 | 5-(3,4-dimethoxyphenyl)-4-[(3-fluorophenyl)amino]nicotinonitrile |
| 103 | 4-anilino-5-(3,4-dimethoxyphenyl)nicotinonitrile |
| 104 | 4-[(2,5-difluorophenyl)amino]-5-(3,4-dimethoxyphenyl)nicotinonitrile |
| 105 | 5-(3,4-dimethoxyphenyl)-4-[(3,4-dimethoxyphenyl)amino]nicotinonitrile |
| 106 | 4-[(4-chloro-2-fluorophenyl)amino]-5-(3,4-dimethoxyphenyl)nicotinonitrile |
| 107 | 4-[(3-chloro-4-fluorophenyl)amino]-5-(3,4-dimethoxyphenyl)nicotinonitrile |
| 108 | 4-[(4-chlorophenyl)amino]-5-(3,4-dimethoxyphenyl)nicotinonitrile |
| 109 | 5-(3,4-dimethoxyphenyl)-4-[(2,4-dimethylphenyl)amino]nicotinonitrile |
| 110 | 5-(3,4-dimethoxyphenyl)-4-[(4-methoxyphenyl)amino]nicotinonitrile |
| 111 | 4-[(3-chloro-4-methoxyphenyl)amino]-5-(3,4-dimethoxyphenyl)nicotinonitrile |
| 112 | 5-(3,4-dimethoxyphenyl)-4-[(4-phenoxy phenyl)amino]nicotinonitrile |
| 113 | 4-[(2,5-dichlorophenyl)amino]-5-(3,4-dimethoxyphenyl)nicotinonitrile |
| 114 | 5-(3,4-dimethoxyphenyl)-4-[(4-methoxy-2-methylphenyl)amino]nicotinonitrile |
| 115 | 4-[(3,4-dichlorophenyl)amino]-5-(3,4-dimethoxyphenyl)nicotinonitrile |
| 116 | 4-[(5-chloro-2-methoxyphenyl)amino]-5-(3,4-dimethoxyphenyl)nicotinonitrile |
| 117 | 4-{[3-(benzyloxy)phenyl]amino}-5-(3,4-dimethoxyphenyl)nicotinonitrile |
| 118 | 5-(3,4-dimethoxyphenyl)-4-[(4-methylphenyl)amino]nicotinonitrile |
| 119 | 5-(3,4-dimethoxyphenyl)-4-[(3,4,5-trimethoxyphenyl)amino]nicotinonitrile |
| 120 | 5-(3,4-dimethoxyphenyl)-4-[(3-phenoxy phenyl)amino]nicotinonitrile |
| 121 | 4-[(2-chloro-5-methoxyphenyl)amino]-5-(3,4-dimethoxyphenyl)nicotinonitrile |
| 122 | 4-({3-chloro-4-[(3-cyanobenzyl)oxy]phenyl}amino)-5-(3,4-dimethoxyphenyl) |
| | nicotinonitrile |
| 124 | 4-({3-chloro-4-[(3-methylbenzyl)oxy]phenyl}amino)-5-(3,4-dimethoxyphenyl) |
| | nicotinonitrile |
| 125 | 4-[(3-chloro-4-{[3-(dimethylamino)benzyl]oxy}phenyl)amino]-5-(3,4- |
| | dimethoxyphenyl)nicotinonitrile |
| 126 | 4-[(2,4-dichlorophenyl)amino]-5-(3,4-dimethoxyphenyl)nicotinonitrile |
| 127 | N-(3-{[3-cyano-5-(3,4-dimethoxyphenyl)pyridin-4-yl]amino}phenyl) acetamide |
| 128 | N-(3-{[3-cyano-5-(3,4-dimethoxyphenyl)pyridin-4-yl]amino}phenyl)-N- |
| | methylacetamide |
| 129 | N-(3-{[3-cyano-5-(3,4-dimethoxyphenyl)pyridin-4-yl]amino}phenyl) |
| | methanesulfonamide |
| 130 | 5-[4-(dimethylamino)phenyl]-4-[(3-methoxyphenyl)amino]nicotinonitrile |
| 131 | 5-[4-(dimethylamino)phenyl]-4-[(3-fluorophenyl)amino]nicotinonitrile |
| 132 | 4-({3-cyano-5-[4-(dimethylamino)phenyl]pyridin-4-yl}amino)benzoic acid |
| 133 | 4-[(4-cyanophenyl)amino]-5-[4-(dimethylamino)phenyl]nicotinonitrile |
| 134 | 4-[(3,4-difluorophenyl)amino]-5-[4-(dimethylamino)phenyl]nicotinonitrile |
| 135 | 4-[(3-bromophenyl)amino]-5-(3,4-dimethoxyphenyl)nicotinonitrile |
| 136 | 4-{[3-(benzyloxy)-4-chloro phenyl]amino}-5-(3,4-dimethoxyphenyl)nicotinonitrile |
| 137 | 4-[(2,4-dichloro-5-methoxyphenyl)amino]-5-(3,4-dimethoxyphenyl)nicotinonitrile |
| 138 | 4-[(2,4-dichloro-5-ethoxyphenyl)amino]-5-(3,4-dimethoxyphenyl)nicotinonitrile |
| 139 | 4-[(2,4-dichloro-5-propoxyphenyl)amino]-5-(3,4-dimethoxyphenyl)nicotinonitrile |
| 140 | 4-[(5-butoxy-2,4-dichlorophenyl)amino]-5-(3,4-dimethoxyphenyl)nicotinonitrile |
| 141 | 4-{[2,4-dichloro-5-(2-hydroxy ethoxy)phenyl]amino}-5-(3,4-dimethoxyphenyl) |
| | nicotinonitrile |
| 142 | 4-{[4-(benzyloxy)-3-chlorophenyl]amino}-5-(3-nitrophenyl)nicotinonitrile |
| 143 | 4-{[3-chloro-4-(pyridin-2-ylmethoxy)phenyl]amino}-5-(3-nitrophenyl)nicotinonitrile |
| 144 | 4-[(3-chloro-4-fluorophenyl)amino]-5-(3-nitrophenyl)nicotinonitrile |
| 145 | 5-(3-aminophenyl)-4-{[4-(benzyloxy)-3-chlorophenyl]amino}nicotinonitrile |
| 146 | 4-[(3-chloro-4-fluorophenyl)amino]-5-(2-nitrophenyl)nicotinonitrile |
| 147 | 5-(2-aminophenyl)-4-[(3-chloro-4-fluorophenyl)amino]nicotinonitrile |
| 148 | 4-[(2,4-dichloro-5-methoxyphenyl)amino]-5-[4-methoxy-3-(2-methoxy ethoxy) |
| | phenyl]nicotinonitrile |
| 149 | 4-[(2,4-dichloro-5-methoxyphenyl)amino]-5-[3-methoxy-4-(2-methoxy |
| | ethoxy)phenyl]nicotinonitrile |
| 150 | 5-[3-(2-chloroethoxy)phenyl]-4-[(2,4-dichloro-5-methoxyphenyl)amino] |
| | nicotinonitrile |
| 151 | 4-[(2,4-dichloro-5-methoxyphenyl)amino]-5-[3-(2-pyrrolidin-1-ylethoxy)phenyl] |
| | nicotinonitrile |
| 152 | 5-[4-(dimethylamino)phenyl]-4-[(3-nitrophenyl)amino]nicotinonitrile |
| 153 | 5-(3-methoxyphenyl)-4-[(3-nitrophenyl)amino]nicotinonitrile |
| 154 | 5-(3-methoxyphenyl)-4-[(3-methoxyphenyl)amino]nicotinonitrile |
| 155 | 4-[(3-fluorophenyl)amino]-5-(3-methoxyphenyl)nicotinonitrile |
| 156 | 4-{[3-cyano-5-(3-methoxyphenyl)pyridin-4-yl]amino}benzoic acid |
| 157 | 4-[(4-cyanophenyl)amino]-5-(3-methoxyphenyl)nicotinonitrile |
| 158 | 4-[(3,4-difluorophenyl)amino]-5-(3-methoxyphenyl)nicotinonitrile |
| 159 | 5-(3,4-dimethoxyphenyl)-4-[(3-hydroxy phenyl)amino]nicotinonitrile |
| 160 | 5-(3,4-dimethoxyphenyl)-4-{[3-(2-hydroxyethoxy)phenyl]amino}nicotinonitrile |
| 161 | 4-[(3-{[(2S)-2-amino-3-phenyl propyl]-oxy}-phenyl)amino]-5-(3,4- |
| | dimethoxyphenyl)nicotinonitrile |
| 162 | 4-[(2-chloro-5-hydroxyphenyl)amino]-5-(5-formyl-1-benzo thein-2-yl)nicotinonitrile |
| 163 | 4-[(2-chloro-5-hydroxyphenyl)amino]-5-[5-(piperidin-1-ylmethyl)-1-benzothien-2- |
| | yl]nicotinonitrile |
| 164 | 4-{[2-chloro-5-(2-hydroxyethoxy)phenyl[amino}-5-[5-(piperidin-1-ylmethyl)-1- |
| | benzothien-2-yl]nicotinonitrile |
| 165 | 4-[(4-amino-2,3-dimethylphenyl)amino]-5-[5-(piperidin-1-ylmethyl)-1-benzothien-2- |
| | yl]nicotinonitrile |
| 166 | 4-[(4-amino-3-methylphenyl)amino]-5-[5-(piperidin-1-ylmethyl)-1-benzothien-2- |
| | yl]nicotinonitrile |
| 167 | 4-[(2-chloro-5-methoxyphenyl)amino]-5-[5-(piperidin-1-ylmethyl)-1-benzofuran-2- |
| | yl]nicotinonitrile |
| 168 | 4-[(2-chloro-5-methylphenyl)amino]-5-[5-(piperidin-1-ylmethyl)-1-benzofuran-2- |
| | yl]nicotinonitrile |
| 169 | 4-[(5-hydroxy-2-phenoxyphenyl)amino]-5-[5-(piperidin-1-ylmethyl)-1-benzofuran-2- |
| | yl]nicotinonitrile |
| 170 | 4-{[3-(aminomethyl)benzyl]amino}-5-(3,4-dimethoxyphenyl)nicotinonitrile |
| 171 | 4-[(2,4-dichloro-5-hydroxyphenyl)amino]-5-[5-(piperidin-1-ylmethyl)-1-benzofuran- |
| | 2-yl]nicotinonitrile |
| 172 | 4-[(4-methoxy-2-methylphenyl)amino]-5-[5-(piperidin-1-ylmethyl)-1-benzofuran-2- |
| | yl]nicotinonitrile |

Pharmaceutically acceptable salts of the compounds of formula I, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lacetic, maleic, malic, malonic, mandelic, methanesulfonic, mucic, naphthalenesulfonic, nitric, oxalic, pamoic, pantothenic, phosphoric, phthalic, propionic, succinic, sulfuric, tartaric, and toluenesulfonic, as well as other known pharmaceutically acceptable acids.

Esters of the compounds of formula I can include various pharmaceutically acceptable esters known in the art that can be metabolized into the free acid form (e.g., a free carboxylic acid form) in a mammal. Examples of such esters include alkyl esters (e.g., of 1 to 10 carbon atoms), cycloalkyl esters (e.g., of 3-10 carbon atoms), aryl esters (e.g., of 6-14 carbon atoms, including of 6-10 carbon atoms), and heterocyclic analogues thereof (e.g., of 3-14 ring atoms, 1-3 of which can be selected from oxygen, nitrogen, and sulfur heteroatoms), wherein the alcohol residue can include further substituents. In some embodiments, esters of the compounds disclosed herein can be C₁₋₁₀ alkyl esters, such as methyl esters, ethyl esters, propyl esters, isopropyl esters, butyl esters, isobutyl esters, t-butyl esters, pentyl esters, isopentyl esters, neopentyl esters, and hexyl esters; C₃₋₁₀ cycloalkyl esters, such as cyclopropyl esters, cyclopropylmethyl esters, cyclobutyl esters, cyclopentyl esters, and cyclohexyl esters; or aryl esters, such as phenyl esters, benzyl esters, and tolyl esters.

Also provided in accordance with the present teachings are prodrugs of the compounds disclosed herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include acetate, formate, and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington: The Science and Practice of Pharmacy, 20th edition, ed. Alfonso R. Gennaro, Lippincott Williams & Wilkins, Baltimore, Md. (2000), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be useful for treating a pathological condition or disorder in a mammal, for example, a human. As used herein, “treating” refers to partially or completely alleviating and/or ameliorating the condition and/or symptoms thereof. The present teachings accordingly include a method of providing to a mammal a pharmaceutical composition that includes a compound of the present teachings in combination or association with a pharmaceutically acceptable carrier. Compounds of the present teachings can be administered alone or in combination with other therapeutically effective compounds or therapies for the treatment of a pathological condition or disorder. As used herein, “therapeutically effective” refers to a substance or an amount that elicits a desirable biological activity or effect.

The present teachings also include use of the compounds disclosed herein as active therapeutic substances for the treatment of a pathological condition or disorder mediated by a protein kinase such as protein kinase C(PKC) and its theta isoform (PKCθ). The pathological condition or disorder can include inflammatory diseases and autoimmune diseases such as asthma, colitis, multiple sclerosis, psoriasis, arthritis, rheumatoid arthritis, osteoarthritis, and joint inflammation. Accordingly, the present teachings further provide methods of treating these pathological conditions and disorders using the compounds described herein. In some embodiments, the methods include identifying a mammal having a pathological condition or disorder mediated by a protein kinase such as PKC and PKCθ, and providing to the mammal an effective amount of a compound as described herein. In some embodiments, the method includes administering to a mammal a pharmaceutical composition that includes a compound disclosed herein in combination or association with a pharmaceutically acceptable carrier.

The present teachings further include use of the compounds disclosed herein as active therapeutic substances for the prevention and/or inhibition of the pathological condition or disorder listed above. Accordingly, the present teachings further provide methods of preventing and/or inhibiting these pathological conditions and disorders using the compounds described herein. In some embodiments, the methods include identifying a mammal having a pathological condition or disorder mediated by a protein kinase such as PKC and PKCθ, and providing to the mammal an effective amount of a compound as described herein. In some embodiments, the method includes administering to a mammal a pharmaceutical composition that includes a compound disclosed herein in combination or association with a pharmaceutically acceptable carrier.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiinflammatory agents. Oral formulations containing an active compound disclosed herein can include any conventionally used oral form, including tablets, capsules, buccal forms, troches, lozenges and oral liquids, suspensions or solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also comprise a compound as described herein in water or fruit juice, containing appropriate solubilizers or emulsifiers as needed.

Liquid carriers can be used in preparing solutions, suspensions, emulsions, syrups, elixirs, and for inhaled delivery. A compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts thereof, in lotions, creams, foams, patches, suspensions, solutions, and suppositories (rectal and vaginal).

When administered for the treatment or inhibition of a particular disease state or disorder, it is understood that an effective dosage can vary depending upon many factors such as the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases, for example those in which the lung is the targeted organ, it may be desirable to administer a compound directly to the airways of the patient, using devices such as metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these active compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, and esters thereof, in lotions, creams, foams, patches, suspensions, solutions, and suppositories (rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For inflammatory diseases, other active compounds (i.e., other active ingredients or agents) effective in their treatment, and particularly in the treatment of asthma and arthritis, can be administered with active compounds of the present teachings. The other agents can be administered at the same time or at different times than the compounds disclosed herein.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components. The use of the term “include” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

As used herein, a “compound” refers to the compound itself and its pharmaceutically acceptable salts, hydrates and esters, unless otherwise understood from the context of the description or expressly limited to one particular form of the compound, i.e., the compound itself, or a pharmaceutically acceptable salt, hydrate or ester thereof.

As used herein, “halo” or “halogen” refers to fluoro, chloro, bromo, and iodo.

As used herein, “oxo” refers to a double-bonded oxygen (i.e., ═O).

As used herein, as a moiety or part of a moiety, “alkyl” refers to a straight-chain or branched saturated hydrocarbon group. In some embodiments, an alkyl group can have from 1 to 10 carbon atoms (e.g, from 1 to 6 carbon atoms). Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. In some embodiments, alkyl groups can be substituted with up to four independently selected —Y—R⁴, —Y—R⁸ or R¹² groups, where Y, R⁴, R⁸ and R¹² are as described herein. A lower alkyl group typically has up to 6 carbon atoms, i.e., one to six carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, as a moiety or part of a moiety, “alkenyl” refers to a straight-chain or branched alkyl group having one or more carbon-carbon double bonds. In some embodiments, an alkenyl group can have from 2 to 10 carbon atoms (e.g., from 2 to 6 carbon atoms). Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In some embodiments, alkenyl groups can be substituted with up to four independently selected —Y—R⁸ or R¹² groups, where Y, R⁸, and R¹² are as described herein.

As used herein, as a moiety or part of a moiety, “alkynyl” refers to a straight-chain or branched alkyl group having one or more carbon-carbon triple bonds. In some embodiments, an alkynyl group can have from 2 to 10 carbon atoms (e.g., from 2 to 6 carbon atoms). Examples of alkynyl groups include ethynyl, propynyl, butynyl, pentynyl, and the like. The one or more carbon-carbon triple bonds can be internal (such as in 2-butyne) or terminal (such as in 1-butyne). In some embodiments, alkynyl groups can be substituted with up to four independently selected —Y—R⁸ or R¹² groups, where Y, R⁸, and R¹² are as described herein.

As used herein, “alkoxy” refers to an —O-alkyl group. In some embodiments, an alkoxy group can have from 1 to 10 carbon atoms (e.g., from 1 to 6 carbon atoms). Examples of alkoxy groups include methoxy, ethoxy, propoxy (e.g., n-propoxy and isopropoxy), t-butoxy groups, and the like.

As used herein, “alkylthio” refers to an —S-alkyl group. Examples of alkylthio groups include methylthio, ethylthio, propylthio (e.g., n-propylthio and isopropylthio), t-butylthio groups, and the like.

As used herein, “haloalkyl” refers to an alkyl group having one or more halogen substituents. In some embodiments, a haloalkyl group can have from 1 to 10 carbon atoms (e.g., from 1 to 6 carbon atoms). Examples of haloalkyl groups include CF₃, C₂F₅, CHF₂, CH₂F, CCl₃, CHCl₂, CH₂C₁, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcaryl, adamantyl, and spiro[4,5]decanyl groups, as well as their homologs, isomers, and the like. In some embodiments, cycloalkyl groups can be substituted with up to four independently selected —Y—R⁴, —Y—R⁸ or R¹² groups, where Y, R⁴, R⁸, and R¹² are as described herein. For example, cycloalkyl groups can include substitution of one or more oxo groups.

As used herein, “heteroatom” refers to an atom of any element other than carbon or hydrogen and includes, for example, nitrogen, oxygen, sulfur, phosphorus, and selenium.

As used herein, “cycloheteroalkyl” refers to a non-aromatic cycloalkyl group that contains at least one ring heteroatom selected from O, N and S, which may be the same or different, and optionally contains one or more double or triple bonds. A cycloheteroalkyl group, as a whole, can have, for example, from 3 to 14 ring atoms and contains from 1 to 5 ring heteroatoms (e.g., from 3-7 ring atoms for a monocyclic cycloheteroalkyl group and from 7 to 14 ring atoms for a polycyclic cycloheteroalkyl group). One or more N or S atoms in a cycloheteroalkyl ring may be oxidized (e.g., morpholine N-oxide, thiomorpholine S-oxide, thiomorpholine S,S-dioxide). In some embodiments, nitrogen atoms of cycloheteroalkyl groups can bear a substituent, for example, a —Y—R⁸ group or an R¹² group, where Y, R⁸, and R¹² as described herein. Cycloheteroalkyl groups can also contain one or more oxo groups, such as piperidone, oxazolidinone, pyrimidine-2,4(1H,3H)-dione, pyridin-2(1H)-one, and the like. Examples of cycloheteroalkyl groups include, among others, morpholine, thiomorpholine, pyran, imidazolidine, imidazoline, oxazolidine, pyrazolidine, pyrazoline, pyrrolidine, pyrroline, tetrahydrofuran, tetrahydrothiophene, piperidine, piperazine, and the like. In some embodiments, cycloheteroalkyl groups can be optionally substituted with up to four independently selected —Y—R⁴, —Y—R⁸ or R¹² groups, where Y, R¹, R⁸, and R¹² are as described herein.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have from 6 to 14 carbon atoms in its ring system, which can include multiple fused rings. In some embodiments, a polycyclic aryl group can have from 8 to 14 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic) and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, aryl groups optionally contain up to four independently selected R⁴, —Y—R⁴, —O—Y—R⁸, or R¹² groups, where Y, R⁴, R⁸, and R¹² are as described herein.

As used herein, “heteroaryl” refers to an aromatic monocyclic ring system containing at least 1 ring heteroatom selected from oxygen (O), nitrogen (N) and sulfur (S) or a polycyclic ring system where at least one of the rings present in the ring system is aromatic and contains at least 1 ring heteroatom. When more than one ring heteroatoms are present they may be the same or different. Polycyclic heteroaryl groups include two or more heteroaryl rings fused together and monocyclic heteroaryl rings fused to one or more aromatic carbocyclic rings, non-aromatic carbocyclic rings, and/or non-aromatic cycloheteroalkyl rings. A heteroaryl group, as a whole, can have, for example, from 5 to 14 ring atoms and contain 1-5 ring heteroatoms. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

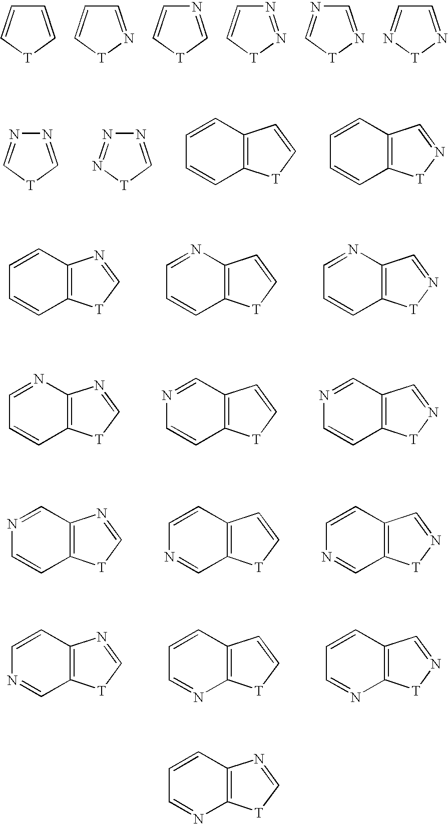

wherein T is O, S, NH, N—Y—R⁴, N—Y—R⁸, or NR¹²; and Y, R⁴, R⁸, and R¹² are as described herein. Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted with up to four substituents independently selected from R⁴, —Y—R⁴, —O—Y—R⁴, —Y—R⁸, or R¹² groups, where Y, R⁴, R⁸, and R¹² are as described herein.

The compounds of the present teachings can include a “divalent group” defined herein as a linking group capable of forming a covalent bond with two other moieties. For example, compounds described herein can include a divalent C₁₋₁₀ alkyl group, such as, for example, a methylene group.

At various places in the present specification, substituents of compounds are disclosed in groups or in ranges. It is specifically intended that the description include each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of other examples, the term “5-14 membered heteroaryl group” is specifically intended to individually disclose a heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, and 13-14 ring atoms; and the phrase “optionally substituted with 1-4 substituents” is specifically intended to individually disclose a chemical group that can include 0, 1, 2, 3, 4, 0-4, 0-3, 0-2, 0-1, 1-4, 1-3, 1-2, 2-4, 2-3, and 3-4 substituents.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure and stereoisomers, as well as other mixtures of the R and S stereoisomers and pharmaceutically acceptable salts thereof. Optical isomers can be obtained in pure form by standard procedures known to those skilled in the art, which include diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include column chromatography, thin-layer chromatography, and high-performance liquid chromatography.

Throughout the specification, structures may or may not be presented with chemical names. Where any question arises as to nomenclature, the structure prevails.

An aspect of the present teachings relates to methods of preparing the compounds disclosed herein. The compounds of the present teachings can be prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, and/or by chromatography such as high performance liquid chromatography (HPLC) or thin layer chromatography.

Preparation of compounds can involve the protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, the entire disclosure of which is incorporated by reference herein for all purposes.

The reactions of the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Scheme 1 below depicts an exemplary synthetic route for the preparation of an intermediate of compounds of formula I.

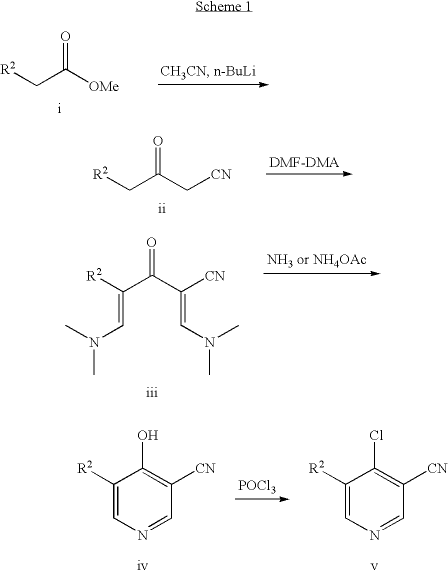

Acetic acid ester i is converted to 3-oxo-butyronitrile ii by reaction with the anion of acetonitrile prepared by reaction of acetonitrile (CH₃CN) with a strong base such as n-butyl lithium (n-BuLi) in a solvent such as THF. Reaction of oxo-butyronitrile ii with dimethylformamide-dimethyl acetal (DMF-DMA) in a solvent such as DMF at high temperature (e.g., 122° C.) results in the formation of bisdimethylaminomethylene intermediate iii which is converted to 4-hydroxy-nicotinonitrile iv by reaction with ammonia (NH₃) or ammonium acetate (NH₄OAc) in a solvent such as ethanol at reflux. Reaction of the hydroxypyridine with refluxing phosphorous oxychloride (POCl₃) with or without catalytic DMF for 2 to 6 hours results in conversion to 4-chloro-nicotinonitrile v.

Scheme 2 below shows an alternative procedure for the preparation of 3-oxo-butyronitrile ii. This alternative procedure involves conversion of acetic acid vi to the corresponding acid chloride by reaction with a chlorinating agent such as thionyl chloride (SOCl₂) followed by reaction of the anion of tert-butylcyanoacetate prepared by reaction of tert-butylcyanoacetate with a base such as sodium hydride (NaH) in a solvent such as THF to give 2-cyano-3-oxo-butanoic acid tert-butyl ester vii, which undergoes deprotection of the ester and decarboxylation to give 3-oxo-butyronitrile ii by reaction with an acid such as trifluoroacetic acid (TFA).

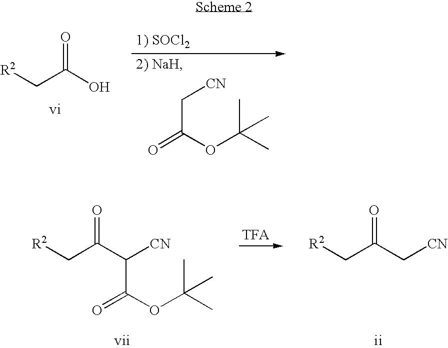

Alternatively, as shown in Scheme 3 below, the bisdimethylaminemethylene intermediate Ic obtained by reaction of 3-oxo-butyronitrile ii with DMF-DMA can be reacted with 3,4-dimethoxybenzylamine at reflux in a solvent such as toluene to give 1-(3,4-dimethoxybenzyl)-4-oxo-1,4-dihydro-pyridine-3-carbonitrile viii. Reaction of viii with excess LiCl in refluxing POCl₃ results in removal of the dimethoxybenzyl group and conversion to the corresponding 4-chloro-nicotinonitrile v.

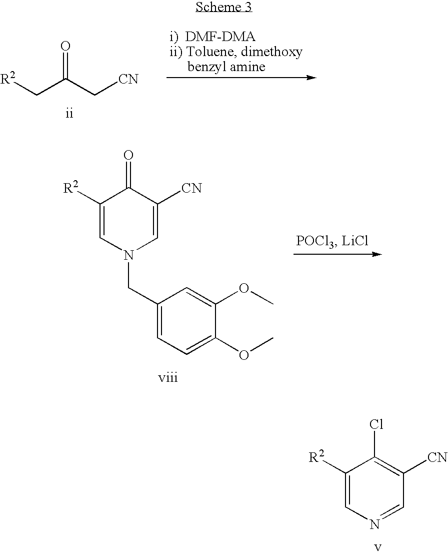

Scheme 4 below depicts an exemplary synthetic route for the preparation of compounds of formula I.

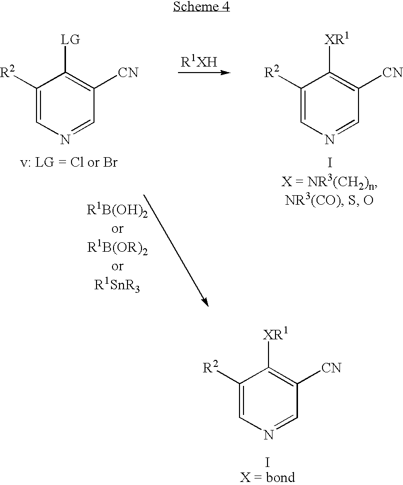

To prepare compounds of formula I where X is —NR³—(CH₂)ₙ—, —NR³(CO)—, —O—, or —S—, where n=0-10, a C-5 substituted 4-chloro-3-cyanopyridine v can be reacted with R¹XH under one of the following reaction conditions: 1) in a solvent such as ethanol (EtOH), propanol, butanol, 2-ethoxyethanol (EtEtOH), 2-methoxyethanol, or 2-butoxyethanol at elevated temperature of 60-180° C., optionally in the presence of pyridine hydrochloride (Pyr.HCl); 2) using an alkali base such as sodium hydride (NaH) in a solvent such as tetrahydrofuran (THF) or dimethylformamide (DMF) at elevated temperatures of 60-120° C.; 3) using a palladium catalyst such as tris(dibenzylidene)acetone dipalladium (Pd₂(dba)₃) and a phosphine ligand such as 2-dicyclohexylphosphino-2′-(N,N-dimethylamino)biphenyl (DavePhos) or tributylphosphine, in the presence of a base such as potassium phosphate (K₃PO₄) or potassium t-butoxide at elevated temperatures of 80-150° C.; 4) using an organic base such as triethylamine (TEA), pyridine, or diisopropylethylamine (DIEA) in a solvent such as DMF, N-methyl-2-pyrrolidone (NMP) or EtEtOH at elevated temperatures of 80-150° C.; 5) using an inorganic base such as cesium carbonate (Cs₂CO₃) in a solvent such as acetonitrile (CH₃CN) or DMF at elevated temperatures of 80-150° C.

When X is a covalent bond, compounds of formula I can be prepared by a coupling reaction of C-5 substituted 4-chloro-3-cyanopyridine v with a boronic acid of formula R¹B(OH)₂, or boronic ester of formula R¹B(OR)₂, where R is an alkyl group (e.g., a lower alkyl group), mediated by a palladium catalyst such as tetrakis(triphenylphosphine)-palladium (0) [(Ph₃P)₄Pd] or palladium (II) acetate (Pd(OAc)₂) in a solvent such as a mixture of dimethoxyethane(DME) and aqueous sodium bicarbonate (aq. NaHCO₃) or aqueous sodium carbonate (aq. Na₂CO₃), optionally in the presence of a phosphine ligand such as triphenyl phosphine (Ph₃P). Alternatively, 4-chloro-3-cyanopyridine v can be treated with a stannane R¹SnR₃, wherein R is an alkyl group (e.g., a lower alkyl group), to yield compounds of formula I.

Referring to Scheme 5 below, additional compounds of formula I where R¹ is substituted with an R⁴ group selected from an aryl group, a heteroaryl group, an alkenyl group and an alkynyl group (formula Ib) can be prepared from compounds of formula I where R² is substituted with a leaving group (LG) such as bromide (Br), iodide (I), chloride (Cl) or trifluoromethane sulfonate (OTf) (formula Ia) as described in Scheme 5 below.

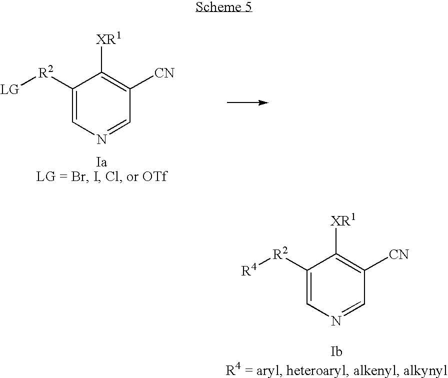

More specifically, compounds of formula Ib where R⁴ is an aryl group or a heteroaryl group can be prepared by treatment of compounds of formula Ia with a boronic acid (R⁴B(OH)₂), a boronic ester (R⁴B(OR)₂, where R is a lower alkyl group) or with an organic stannane reagent (e.g., R⁴SnBu₃) mediated by a palladium catalyst (e.g., (Ph₃P)₄Pd or Pd(OAc)₂) in a solvent such as a mixture of DME and aq. NaHCO₃ or aq. Na₂CO₃, optionally in the presence of a phosphine ligand such as Ph₃P.

Similarly, compounds of formula Ib where R⁴ is an alkenyl group or an alkynyl group can be prepared by treating compounds of formula Ia with an alkene or alkyne of formula R⁴—H or with a boronic acid or ester or an organic stannane reagent in the presence of a palladium catalyst (e.g., (Ph₃P)₄Pd, dichlorobis(triphenylphosphine)palladium (II), or Pd(OAc)₂) in a solvent such as DMF, NMP, dioxane, or DME, in the presence of a ligand such as Ph₃P or tri-o-tolylphosphine and a base (e.g., potassium carbonate (K₂CO₃) or Na₂CO₃), optionally with the addition of an organic base such as TEA. A catalytic amount of copper(I) iodide can be optionally used for this coupling reaction.

Scheme 6 depicts a synthetic route for preparing additional compounds of formula I where both R² and R⁴ are aryl or heteroaryl groups and R⁴ is further substituted with an amide (formula Id).

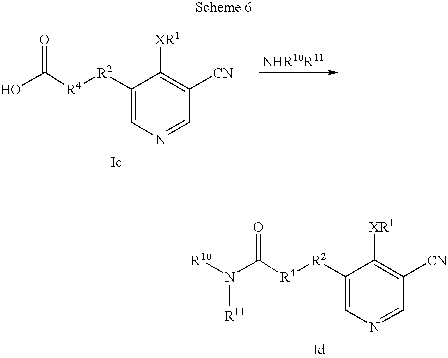

Compounds of formula I where R² is substituted by an aryl or heteroaryl group substituted by a carboxylic acid (formula Ic) can be treated with an amine of formula NHR¹⁰R¹¹ in the presence of a catalyst (e.g., benzotriazol-1-yloxytris(dimethyl amino)phosphonium hexafluorophosphate (BOP)) and an organic amine (e.g., TEA, DIEA, or pyridine) in a solvent such as MeOH or EtOH at ambient temperature to elevated temperatures of 50-80° C. to provide compounds of formula Id as described.

Additional compounds of formula I where R² is substituted with —O—Y—NR⁶R⁷ (formula If) can be prepared as depicted in Scheme 7 below, by treating compounds of formula I where R² is substituted with —O—Y-LG (formula Ie), where LG is Cl, Br, methanesulfonyl (mesyl, OMs), or p-toluenesulfonyl (tosyl, OTs), with an amine of formula NHR⁶R⁷ in a solvent such as EtOH, DME or DMF optionally in the presence of NaI or a base such as K₂CO₃.

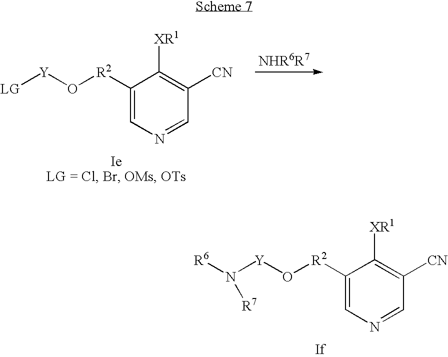

As depicted in Scheme 8, compounds of formula I wherein R² is substituted by —CH₂—NR⁶YR⁷ (formula Ih) can be prepared by treating compounds of formula I where R² contains an aldehyde functionality (formula Ig) with an amine of formula HNR⁶YR⁷ in the presence of a reducing agent (e.g., sodium triacetoxyborohydride (Na(OAc)₃BH) or sodium cyanoborohydride) in a solvent such as dichloromethane (CH₂Cl₂) or THF with the optional addition of DMF or NMP and preferably in the presence of acetic acid. Compounds of formula I wherein R² is substituted by —CH₂—OH (formula Ii) can be formed as a by-product of this reductive amination reaction.

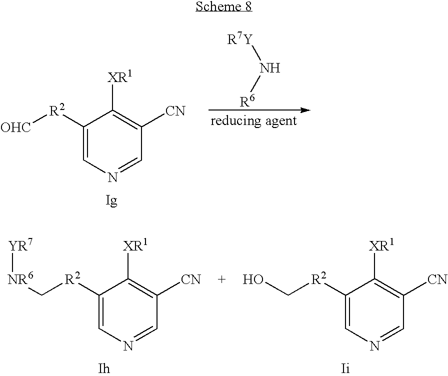

As depicted in Scheme 9, compounds of formula I where R² is substituted by —OYR⁵ (formula Ik) can be prepared by treating compounds of formula I where R² contains a hydroxyl functionality (formula Ij) with an alcohol of formula R⁵YOH under Mitsunobu conditions. This reaction can be conducted in a solvent such as THF in the presence of Ph₃P and either diethyl azodicarboxylate or di-t-butyl azodicarboxylate.

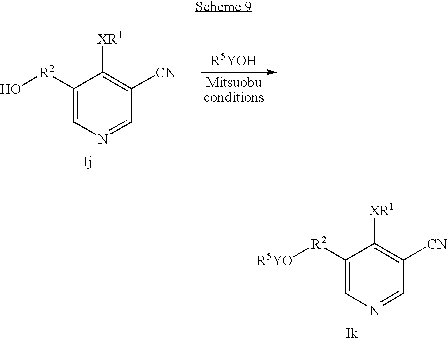

Additional compounds of formula I wherein X is not a bond can be prepared as shown in Scheme 10, Scheme 11, and Scheme 12 below.

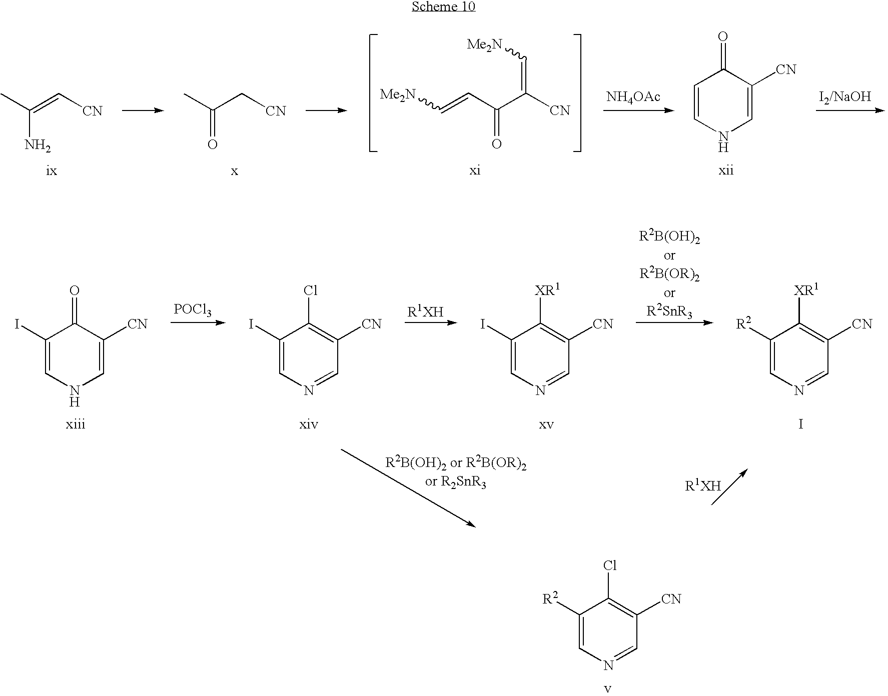

A mixture of 3-aminobut-2-enenitrile ix is heated in acid (e.g., aqueous HCl) to yield acetoacetonitrile x. Acetoacetonitrile x is treated with t-butoxybis(dimethyl amino)methane and DMF-DMA at an elevated temperature to yield 5-(dimethylamino)-2-[(dimethylamino)methylene]-3-oxopent-4-enenitrile xi, which is then treated with ammonium acetate in EtOH at reflux to produce 4-hydroxynicotinonitrile xii. (An alternate synthesis of 4-hydroxynicotinonitrile was reported in the literature: Broekman, F. W. et al., Recueil des Travaux Chimiques des Pays-Bas, 81: 792-796 (1962)). A mixture of 4-hydroxynicotinonitrile xii, iodine and NaOH in water is heated overnight to yield 4-hydroxy-5-iodonicotinonitrile xiii, which is then treated with POCl₃ at an elevated temperature to yield 4-chloro-5-iodonicotinonitrile xiv. Intermediate xiv can then be treated with R¹XH, wherein X is not a bond (e.g., R¹NH₂, R¹OH, R¹SH, etc.) to yield the 4-substituted 5-iodo-nicotinonitrile xv. Further treatment with a boronic acid R²B(OH)₂, boronic acid ester R²B(OR)₂ or stannane R²SnR₃ (where R, in each case, is a lower alkyl group) yields compounds of formula I. Alternatively, intermediate xiv can be treated with a boronic acid R²B(OH)₂, a boronic acid ester R²B(OR)₂ or a stannane R²SnR₃ (where R, in each case, is a lower alkyl group), followed by a reaction with R¹XH to provide compounds of formula I.

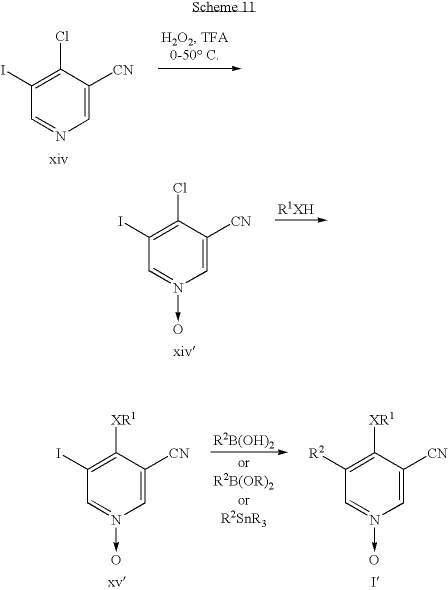

As depicted in Scheme 11, treatment of 4-chloro-5-iodonicotinonitrile xiv with an oxidizing agent, preferably hydrogen peroxide, in trifluoroacetic acid at temperatures of 0-50° C., provides 4-chloro-5-iodo-1-oxy-nicotinonitrile xiv′. Addition of R¹XH under the conditions noted previously provides compounds of formula xv′. Addition of a boronic acid, ester, or an organostannane (where R, in each case, is a lower alkyl group) under the conditions noted previously provides compounds of formula I′.

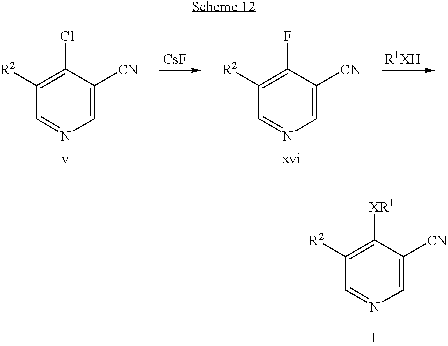

As shown in Scheme 12, treatment of compounds of formula v with CsF in a solvent such as DMF provides the 4-fluoro analog xvi. Subsequent displacement of the 4-fluoro group with R¹XH in a solvent such as DMSO provides compounds of formula I.

Aspects of the present teachings may be further understood in light of the following examples, which should not be construed as limiting the scope of the present teachings in any way.

More specifically, the following examples illustrate various synthetic routes which can be used to prepare compounds of formula I.

### EXAMPLE 1

### Preparation of 4-[(3-chlorophenyl)amino]-5-(3,4-dimethoxyphenyl)nicotinitrile

A solution of 3,4-dimethoxyphenyl acetic acid (50 mM) in methanol (MeOH, 100 mL) with concentrated sulfuric acid (H₂SO₄, 1 mL) or concentrated hydrochloric acid (HCl) was heated at reflux overnight. Concentration to dryness on a rotary evaporator and high vacuum pump overnight gave 3,4-dimethoxy-phenyl)acetic acid methyl ester as an oil which was used directly in the next step.

To a 1.0 L three-necked round-bottomed flask was added 50 mL of THF and the reaction mixture was cooled to −78° C. Butyl lithium (1.6 M, 14.4 mL, 23 mmol) was added dropwise keeping the temperature below −70° C. Acetonitrile (1.3 mL, 25 mmol) in 30 mL of THF was added dropwise to the flask amidst stirring and cooling. After 2 hours of stirring, 3,4-dimethoxy-phenyl)acetic acid methyl ester (2.3 g, 11 mmol) was added to the resulting white colloidal mixture in the flask. The reaction mixture was stirred for a further two hours, followed by the addition of saturated ammonium chloride solution (75 mL) at −78° C. The organic layer was separated, dried with sodium sulfate, filtered to remove the drying agent and evaporated to dryness to give the crude product. This crude product was purified by silica gel column chromatography, eluting with 30-70% ethyl acetate in hexanes to yield 4-(3,4-dimethoxyphenyl)-3-oxo-butyronitrile in the form of a solidifying amber oil, 1.8 g (75%).

To a solution of 4-(3,4-dimethoxyphenyl)-3-oxo-butyronitrile (5.0 g, 23 mmol) in DMF (12 mL) was added DMF-DMA (13.5 mL, 101 mmol) and the solution heated at 122° C. overnight. Concentration on a rotary evaporator under high vacuum gave an orange-red solid. This solid was dissolved in EtOH (100 mL) and excess NH₄OAc was added and the reaction mixture was heated at 85° C. for 1 hour. The reaction mixture was allowed to cool to room temperature (room temperature) for 1 hour then the solids were collected by filtration and washed with EtOH (cold) to give 5-(3,4-dimethoxyphenyl)-4-hydroxynicotinonitrile (4.1 g, 69%) as a brown solid. The filtrate was concentrated on a rotary evaporator and the residue purified on silica gel with 0-25% MeOH in methylene chloride (CH₂Cl₂) to give an additional amount of 5-(3,4-dimethoxyphenyl)-4-hydroxynicotinonitrile.

A solution of 5-(3,4-dimethoxyphenyl)-4-hydroxynicotinonitrile (4 g, 15.7 mmol) in POCl₃ (25 mL) was heated at 125° C. for 1.5 hours, then cooled to room temperature and poured into an ice/3 N sodium hydroxide/ethyl acetate mixture. The mixture was stirred and the layers separated. The organic layer was dried over magnesium sulfate (MgSO₄), filtered and concentrated to give 4-chloro-5-(3,4-dimethoxyphenyl)nicotinonitrile (3.9 g, 91%) as a brown solid.

A solution of 4-chloro-5-(3,4-dimethoxyphenyl)nicotinonitrile (55 mg, 0.2 mmol), 3-chloroaniline (25 mg, 0.2 mmol) and Pyr.HCl (23 mg, 0.2 mmol) in EtOEtOH (2 mL) was heated at reflux for 8 hours, then cooled to room temperature and concentrated. The residue was purified by reverse-phase HPLC to give 5-(3,4-dimethoxyphenyl)-4-[(3-chlorophenyl)amino]nicotinonitrile 101 (3.4 mg). MS: 367 [M+H].

Following procedures analogous to those described for the preparation of compound 101 and using the appropriate aniline in the last step, the compounds in Table 2 were prepared. The HPLC retention times provided in Table 2 as well as in Examples 2-22 below were obtained using conditions as designated below:

(a) Instrument—Agilent 1100; column: Keystone Aquasil C18, from Thermo Fisher Scientific, Inc. (Waltham, Mass.); mobile phase A: 10 mM NH₄OAc in 95% water/5% CH₃CN; mobile phase B: 10 mM NH₄OAc in 5% water/95% CH₃CN; flow rate: 0.800 ml/min.; column temperature: 40° C.;

(b) Column YMC C18, 4.6×500 mm, 5 microns, from YMC (Kyoto, Japan); mobile phase A: 90% water+10% MeOH+0.02% H₃PO₄; mobile phase B: 90% MeOH+10% water+0.02% H₃PO₄; 1-100% B in 2 min., up to 10 min. 100% B, then 100-1% B in 1 min;

(c) Column: Prodigy ODS3, 4.6×150 mm, from Phenomenex (Torrance, Calif.); mobile phase A: 0.02% TFA in water; mobile phase B: 0.02% TFA in CH₃CN; 10-95% B in 20 min.; flow rate: 1.0 mL/min.; column temperature: 40° C.; detection wavelength: 215 nm; and

(d) Column: Aquasil C18, 50×2.1 mm, from Thermo Fisher Scientific, Inc. (Waltham, Mass.); mobile phase A: 0.1% formic acid in water; mobile phase B: 0.1% formic acid in acetonitrile, 0-100% B in 2.5 min., flow rate: 0.8 mL/min.; column temperature: 40° C.; detection wavelength: 254 nm.

TABLE 2

| | | HPLC | |
|---|---|---|---|
| | | Retention | Observed Ion |
| | | Time | m/e |
| Compound | Compound Name | (min.) | [M + H] |
| 102 | 5-(3,4-dimethoxyphenyl)-4-[(3-fluorophenyl) | 2.12^{(a)} | 350 |
| | amino]nicotinonitrile | | |
| 103 | 4-anilino-5-(3,4-dimethoxyphenyl)nicotinonitrile | N/A | 332 |
| 104 | 4-[(2,5-difluorophenyl)amino]-5-(3,4- | 1.95^{(a)} | 368 |
| | dimethoxyphenyl)nicotinonitrile | | |
| 105 | 5-(3,4-dimethoxyphenyl)-4-[(3,4- | 1.60^{(a)} | 392 |
| | dimethoxyphenyl)amino]nicotinonitrile | | |
| 106 | 4-[(4-chloro-2-fluorophenyl)amino]-5-(3,4- | 2.06^{(a)} | 384 |
| | dimethoxyphenyl)nicotinonitrile | 2.06^{(a)} | 384 |
| 107 | 4-[(3-chloro-4-fluorophenyl)amino]-5-(3,4- | 2.00^{(a)} | 384 |
| | dimethoxyphenyl)nicotinonitrile | | |
| 108 | 4-[(4-chlorophenyl)amino]-5-(3,4- | 1.95^{(a)} | 366 |
| | dimethoxyphenyl)nicotinonitrile | | |
| 109 | 5-(3,4-dimethoxyphenyl)-4-[(2,4- | 2.24^{(a)} | 360 |
| | dimethylphenyl)amino]nicotinonitrile | | |
| 110 | 5-(3,4-dimethoxyphenyl)-4-[(4-methoxyphenyl) | 2.09^{(a)} | 362 |
| | amino]nicotinonitrile | | |
| 111 | 4-[(3-chloro-4-methoxyphenyl)amino]-5-(3,4- | 2.17^{(a)} | 396 |
| | dimethoxyphenyl)nicotinonitrile | | |
| 112 | 5-(3,4-dimethoxyphenyl)-4-[(4-phenoxyphenyl) | 2.41^{(a)} | 424 |
| | amino]nicotinonitrile | | |
| 113 | 4-[(2,5-dichlorophenyl)amino]-5-(3,4- | 2.35^{(a)} | 400 |
| | dimethoxyphenyl)nicotinonitrile | | |
| 114 | 5-(3,4-dimethoxyphenyl)-4-[(4-methoxy-2- | 1.85^{(a)} | 376 |
| | methylphenyl)amino]nicotinonitrile | | |
| 115 | 4-[(3,4-dichlorophenyl)amino]-5-(3,4- | 2.36^{(a)} | 400 |
| | dimethoxyphenyl)nicotinonitrile | | |
| 116 | 4-[(5-chloro-2-methoxyphenyl)amino]-5-(3,4- | 2.12^{(a)} | 396 |
| | dimethoxyphenyl)nicotinonitrile | | |
| 117 | 4-{[3-(benzyloxy)phenyl]amino}-5-(3,4- | 2.36^{(a)} | 438 |
| | dimethoxyphenyl)nicotinonitrile | | |
| 118 | 5-(3,4-dimethoxyphenyl)-4-[(4-methylphenyl) | 1.94^{(a)} | 346 |
| | amino]nicotinonitrile | | |
| 119 | 5-(3,4-dimethoxyphenyl)-4-[(3,4,5- | 1.77^{(a)} | 422 |
| | trimethoxyphenyl)amino]nicotinonitrile | | |
| 120 | 5-(3,4-dimethoxyphenyl)-4-[(3-phenoxyphenyl) | 2.38^{(a)} | 424 |
| | amino]nicotinonitrile | | |
| 121 | 4-[(2-chloro-5-methoxyphenyl)amino]-5-(3,4- | 2.78^{(a)} | 396 |
| | dimethoxyphenyl)nicotinonitrile | | |
| 122 | 4-({3-chloro-4-[(3-cyanobenzyl)oxy]phenyl} | 2.35^{(a)} | 497 |
| | amino)-5-(3,4-dimethoxyphenyl)nicotinonitrile | | |
| 123 | 4-{[3-chloro-4-(thien-2-ylmethoxy)phenyl]amino}- | 2.37^{(a)} | 478 |
| | 5-(3,4-dimethoxyphenyl)nicotinonitrile | | |
| 124 | 4-({3-chloro-4-[(3-methylbenzyl)oxy]phenyl} | N/A | 487 |
| | amino)-5-(3,4-dimethoxyphenyl)nicotinonitrile | | |
| 125 | 4-[(3-chloro-4-{[3-(dimethylamino)benzyl]oxy} | 2.28^{(a)} | 515 |
| | phenyl)amino]-5-(3,4-dimethoxyphenyl) | | |
| | nicotinonitrile | | |
| 126 | 4-[(2,4-dichlorophenyl)amino]-5-(3,4- | 2.16^{(a)} | 400 |
| | dimethoxyphenyl)nicotinonitrile | | |
| 127 | N-(3-{[3-cyano-5-(3,4-dimethoxyphenyl)pyridin-4- | 1.84^{(a)} | 389.2 |
| | yl]amino}phenyl)acetamide | | |
| 128 | N-(3-{[3-cyano-5-(3,4-dimethoxyphenyl)pyridin-4- | 2.76^{(a)} | 402.7 |
| | yl]amino}phenyl)-N-methylacetamide | | |
| 129 | N-(3-{[3-cyano-5-(3,4-dimethoxyphenyl)pyridin-4- | 1.86^{(a)} | 425.1 |
| | yl]amino}phenyl)methanesulfonamide | | |
| 130 | 5-[4-(dimethylamino)phenyl]-4-[(3- | 3.0^{(b)} | 345.4 |
| | methoxyphenyl)amino]nicotinonitrile | | |
| 131 | 5-[4-(dimethylamino)phenyl]-4-[(3-fluorophenyl) | 3.4^{(b)} | 333.4 |
| | amino]nicotinonitrile | | |
| 132 | 4-({3-cyano-5-[4-(dimethylamino)phenyl]pyridin- | 3.0^{(b)} | 356.8 [M − H] |
| | 4-yl}amino)benzoic acid | | |
| 133 | 4-[(4-cyanophenyl)amino]-5-[4-(dimethylamino) | 3.3^{(b)} | 340.2 |
| | phenyl]nicotinonitrile | | |
| 134 | 4-[(3,4-difluorophenyl)amino]-5-[4-(dimethylamino) | 3.2^{(b)} | 351.2 |
| | phenyl]nicotinonitrile | | |

### EXAMPLE 2

### Preparation of 4-[(3-bromophenyl)amino]-5-(3,4-dimethoxyphenyl)nicotinonitrile 135

A mixture of 4-chloro-5-(3,4-dimethoxyphenyl)nicotinonitrile (0.5 g, 1.82 mmol), 3-bromoaniline (0.313 g, 1.82 mmol), and 0.05 g of Pyr.HCl in 8 ml of EtOEtOH was heated at reflux for 8 hours. The solid was collected and dissolved in a mixture of saturated sodium bicarbonate (NaHCO₃) and CH₂Cl₂. The layers were separated and the organic layer was dried with MgSO₄ and filtered through a pad of Magnasol®. Solvent was removed and the residue was recrystallized from iso-propanol/hexane to give 0.43 g of 4-[(3-bromophenyl)amino]-5-(3,4-dimethoxyphenyl)nicotinonitrile 135. HPLC retention time^{(a)}: 2.72 min.; MS: 410.2 m/e (M+H).

### EXAMPLE 3

### Preparation of 4-{[3-(benzyloxy)-4-chlorophenyl]amino}-5-(3,4-dimethoxyphenyl)nicotinonitrile 136

This compound was prepared from 4-chloro-5-(3,4-dimethoxyphenyl)nicotinonitrile and 3-benzyloxy-4-chloroaniline using procedures analogous to those described in Example 2. HPLC retention time^{(a)}: 2.90 min.; MS: 470.2 m/e (M+H).

### EXAMPLE 4

### Preparation of 4-[(2,4-dichloro-5-methoxyphenyl)amino]-5-(3,4-dimethoxyphenyl)nicotinonitrile 137

A mixture of 4-chloro-5-(3,4-dimethoxyphenyl)nicotinonitrile (0.5 g, 1.82 mmol), 2,4-dichloro-5-methoxyaniline (0.402 g, 2.1 mmol), Pd₂(dba)₃ (0.167 g, 0.18 mmol), 2-dicyclohexylphosino-2′-(N,N-dimethylamino)biphenyl (0.22 g, 0.56 mmol), and K₃PO₄ (0.58 g, 2.73 mmol) in 10 ml of DME was heated at reflux for 45 minutes. The hot mixture was filtered and solids were washed with ether. The combined filtrates were washed with saturated NaHCO₃, dried (MgSO₄), and filtered through a pad of Magnesol®. Solvent was removed and the residue was chromatographed on silica gel. Product was eluted with CH₂Cl₂-ether and then recrystallized from iso-propanol/hexane giving 0.21 g of 4-[(2,4-dichloro-5-methoxyphenyl)amino]-5-(3,4-dimethoxyphenyl)nicotinonitrile 137. HPLC retention time^{(a)}: 0.86 min.; MS: 430.2 m/e (M+H).

Following procedures analogous to those described for the preparation of compound 137 and using the appropriate aniline, the compounds in Table 3 were prepared.

TABLE 3

| | | HPLC | Observed | | |
|---|---|---|---|---|---|
| | | Retention | Ion | | |
| | | Time | m/e | M.P. | |
| Compound | Compound Name | (min) | [M + H] | (° C.) | Appearance |
| 138 | 4-(2,4-dichloro-5-ethoxyphenyl | | 444.1 | 125-126 | white solid |
| | amino)-5-(3,4-dimethoxy-phenyl)- | | | | |
| | nicotinonitrile | | | | |
| 139 | 4-[(2,4-dichloro-5-propoxy | 2.35^{(a)} | 458.1 | N/A | light yellow |
| | phenyl)amino]-5-[(3,4-dimethoxy | | | | foam |
| | phenyl)amino]nicotinonitrile | | | | |
| 140 | 4-[(5-butoxy-2,4-dichlorophenyl) | 2.47^{(a)} | 472.1 | N/A | light tan |
| | amino]-5-(3,4-dimethoxy phenyl) | | | | foam |
| | nicotinonitrile | | | | |
| 141 | 4-{[2,4-dichloro-5-(2-hydroxy | 2.35^{(a)} | 460.1 | N/A | off-white |
| | ethoxy)phenyl]amino}-5-(3,4- | | | | solid |
| | dimethoxyphenyl)nicotinonitrile | | | | |

### EXAMPLE 5

### Preparation of 4-{[4-(benzyloxy)-3-chlorophenyl]amino}-5-(3-nitrophenyl)nicotinonitrile 142

3-Nitrophenylacetic acid (9.5 g, 52 mmol) and SOCl₂ (20 mL) were stirred overnight at room temperature, then evaporated to dryness. In a separate flask NaH (60% dispersion in oil, 5.5 g, 1.4 mmol) was suspended in THF (100 mL). The mixture was cooled to 0° C. and tert-butylcyanoacetate (8.8 g, 62 mmol) was added. After 15 minutes, a solution of 3-nitrophenylacetyl chloride from above in THF was added dropwise. The cooling bath was removed and the mixture allowed to warm to room temperature and stirred for 4 hours. The reaction mixture was quenched by the addition of brine, and extracted with ethyl acetate (EtOAc, 2×200 mL). The combined organic extracts were dried over MgSO₄ and concentrated. The crude 2-cyano-4-(3-nitrophenyl)-3-oxo-butyric acid tert-butyl ester was used in the next step without further purification.

To a solution of 2-cyano-4-(3-nitro-phenyl)-3-oxo-butyric acid tert-butyl ester (9.5 g, 31 mmol) in toluene (40 mL) was added TFA (4 mL) and the solution heated at reflux for 2 hours, then the solvent was evaporated in vacuo. The residue was purified by silica gel flash chromatography to give 4-(3-nitrophenyl)-3-oxo-butyronitrile (4.0 g, 37% over 2 steps).

Following procedures analogous to those described in Example 1,4-(3-nitrophenyl)-3-oxo-butyronitrile was converted to 4-hydroxy-5-(3-nitrophenyl)nicotinonitrile, which was then converted to 4-chloro-5-(3-nitrophenyl)nicotinonitrile.

A solution of 4-chloro-5-(3-nitrophenyl)nicotinonitrile (2.1 g, 8.1 mmol) and 4-benzyloxy-3-chloroaniline (1.89 g, 8.1 mmol) in 50 ml of EtOEtOH was heated at reflux for 6.5 hours. The mixture was cooled and diluted with 450 ml of ether. A solution of HCl in 10 ml of ether was added. The solid was collected by filtration and washed with ether. The solid was suspended in saturated NaHCO₃ and the mixture was stirred with EtOAc until solids dissolved. The organic layer was dried (MgSO₄) and filtered through a pad of Magnesol®. Solvent was removed and the residue was recrystallized from EtOH giving 2.15 g of 4-{[4-(benzyloxy)-3-chlorophenyl]amino}-5-(3-nitrophenyl)nicotinonitrile 142. HPLC retention time (a): 3.06 min.; MS: 456.8 m/e (M+H).

Following procedures analogous to those described for the preparation of compound 142 and using the appropriate aniline, the compounds in Table 4 were prepared.

TABLE 4

| | | HPLC | Observed | | |
|---|---|---|---|---|---|
| | | Retention | Ion | Melting | |
| | | Time | m/e | Range | |
| Compound | Compound Name | (min) | [M + H] | (° C.) | Appearance |
| 143 | 4-{[3-chloro-4-(pyridin-2- | 2.74^{(a)} | 458.1 | 183-185 | tan solid |
| | ylmethoxy)phenyl]amino}-5-(3- | | | | |
| | nitrophenyl)nicotinonitrile | | | | |
| 144 | 4-[(3-chloro-4- | 2.84^{(a)} | 369 | 165-175 | tan solid |
| | fluorophenyl)amino]-5-(3- | | | | |
| | nitrophenyl)nicotinonitrile | | | | |

### EXAMPLE 6

### Preparation of 5-(3-aminophenyl)-4-{[4-(benzyloxy)-3-chlorophenyl]amino}nicotinonitrile 145

A mixture of 4-{[4-(benzyloxy)-3-chlorophenyl]amino}-5-(3-nitrophenyl)nicotinonitrile 142 (2.0 g, 4.38 mmol), iron (1.47 g, 26.3 mmol), and acetic acid (ACOH, 1.58 g, 26.3 mmol) in 90 mL of MeOH was stirred at reflux for 3 hours. The hot mixture was filtered, and the solids collected were washed with hot THF. The combined organic solutions were concentrated and then redissolved in a hot THF-ethyl acetate mixture. The suspension was filtered and washed with brine/saturated NaHCO₃. The organic layer was dried (MgSO₄) and filtered through a pad of Magnesol®. Solvent was removed giving 1.81 g of 5-(3-aminophenyl)-4-{[4-(benzyloxy)-3-chlorophenyl]amino}nicotinonitrile 145. HPLC retention time (a): 2.74 min.; MS: 426.8 m/e (M+H).

### EXAMPLE 7

### Preparation of 4-[(3-chloro-4-fluorophenyl)amino]-5-(2-nitrophenyl)nicotinonitrile 146

4-Chloro-5-(2-nitrophenyl)nicotinonitrile was prepared from 2-nitrophenyl acetic acid using procedures analogous to those described for the preparation of 4-chloro-5-(3-nitrophenyl)nicotinonitrile in Example 5. MS: 260.1 m/e (M+H).

A mixture of 4-chloro-5-(2-nitrophenyl)nicotinonitrile (4 g, 15.41 mmol), Pyr.HCl (0.89 g, 7.7 mmol), and 3-chloro-4-fluoroaniline (2.8 g, 19.26 mmol) in 15 ml of diglyme was heated at 130° C. for 27 hours. The mixture was cooled and ethereal HCl was added and solids were collected. The solid was stirred with saturated NaHCO₃ and CH₂Cl₂ until it dissolved. The solution washed with brine, dried (MgSO₄), and filtered through a pad of Magnesol® and concentrated. The residue was chromatographed on silica gel to give 2.3 g of 4-[(3-chloro-4-fluorophenyl)amino]-5-(2-nitrophenyl)nicotinonitrile 146. HPLC retention time^{(a)}: 3.58 min.; MS: 369.1 m/e (M+H).

### EXAMPLE 8

### Preparation of 5-(2-aminophenyl)-4-[(3-chloro-4-fluorophenyl)amino]nicotinonitrile 147

5-(2-Aminophenyl)-4-[(3-chloro-4-fluorophenyl)amino]nicotinonitrile 147 was prepared by reducing 4-[(3-chloro-4-fluorophenyl)amino]-5-(2-nitrophenyl)nicotinonitrile 146 as described above in Example 6. HPLC retention time^{(a)}: 2.06 min.; MS: 339.2 m/e (M+H).

### EXAMPLE 9

### Preparation of 4-[(2,4-dichloro-5-methoxyphenyl)amino]-5-[4-methoxy-3-(2-methoxyethoxy)phenyl]nicotinonitrile 148

To a stirred solution of 3-hydroxy-4-methoxyphenyl acetic acid (24.84 g, 136 mmol) in 0.2 L of MeOH was added 1 mL of H₂SO₄ and heated at reflux overnight. The methanol was evaporated in vacuo and the residue poured into saturated NaHCO₃ solution and extracted with EtOAc (3×150 mL). Combined organic extracts were then washed with brine, dried over anhydrous MgSO₄, filtered, and concentrated in vacuo to yield 23.94 g (90%) of (3-hydroxy-4-methoxy-phenyl)-acetic acid methyl ester as a yellow oil.

To a stirred solution of 3-hydroxy-4-methoxyphenyl acetic acid methyl ester (5 g, 25.48 mmol), tetrabutylammonium iodide (0.941 g, 2.5 mmol), and 2-bromoethylmethyl ether (4.6 mL, 50.9 mmol) in 150 mL of acetone was added cesium carbonate (17.4 g). The mixture was stirred for 21.5 hours at reflux. The mixture was concentrated and the residue was extracted from water with EtOAc. The combined organic extracts were then dried over anhydrous sodium sulfate (Na₂SO₄), filtered, and concentrated in vacuo to yield 8.15 g of orange oil. The oil was purified by flash chromatography over silica gel using 10-50% EtOAc in hexane as the eluent. Combined product-containing fractions were concentrated to give 5.33 g (82%) of [4-methoxy-3-(2-methoxyethoxy)phenyl]acetic acid methyl ester as a light yellow oil.

To a 250 mL three-necked round-bottomed flask was added 10 mL of anhydrous THF and cooled to −78° C. n-Butyl lithium (2.5 M in hexane, 8.06 mL, 12.9 mmol) was added to the flask and let stir for 5 minutes. Anhydrous acetonitrile (0.696 mL, 13.3 mmol) in 5 mL of anhydrous THF was added drop-wise to the flask with stirring and cooling at −78° C. After 1 hour of stirring, [4-methoxy-3-(2-methoxyethoxy)phenyl]acetic acid methyl ester (1.095 g, 4.3 mmol) in 10 mL of anhydrous THF was added drop-wise to the resulting white colloidal mixture in the flask. The reaction mixture was stirred for an additional 2 hours, followed by the addition of saturated NH₄Cl solution at −78° C. The solution was warmed to room temperature, diluted with 100 mL water and extracted with EtOAc (3×100 mL). The organic layer was separated, washed with brine, dried with anhydrous MgSO₄, filtered, and concentrated in vacuo. The crude product was purified by silica gel chromatography eluting with 30-60% EtOAc in hexanes to yield 769.4 mg (68%) of 4-[4-methoxy-3-(2-methoxy-ethoxy)-phenyl]-3-oxo-butyronitrile as a colorless oil.

To a stirred solution of 4-[4-methoxy-3-(2-methoxyethoxy)phenyl]-3-oxo-butyronitrile (9.91 g, 34.5 mmol) in 20 mL anhydrous DMF was added DMF/DMA (20.2 mL, 152 mmol) and the solution heated at 100° C. for 15 hours. The reaction was concentrated in vacuo and then the crude material was stirred with 3,4-dimethoxy-benzylamine (0.687 mL, 41.4 mmol) in 20 mL of anhydrous toluene at reflux for 2 hours. The reaction was cooled, concentrated in vacuo, and purified by silica gel chromatography eluting with 50-100% EtOAc/hexane to yield 8.5 g (55%) of 1-(3,4-dimethoxybenzyl)-5-[4-methoxy-3-(2-methoxyethoxy)phenyl]-4-oxo-1,4-dihydro-pyridine-3-carbonitrile as a yellow/orange foam.

A solution of 1-(3,4-dimethoxybenzyl)-5-[4-methoxy-3-(2-methoxyethoxy)phenyl]-4-oxo-1,4-dihydro-pyridine-3-carbonitrile (300 mg, 0.666 mmol) and lithium chloride (LiCl, 254 mg, 6 mmol) in 2.5 mL POCl₃ was heated at reflux for 2.5 hours. The excess POCl₃ was removed by concentrating in vacuo and then the residue was co-evaporated with toluene. The residue was dissolved in 100 mL EtOAc and washed with ice-cold 1 N aqueous NaOH. The organic layer was separated, dried over anhydrous MgSO₄, filtered, concentrated in vacuo, and the resulting solid was triturated with isopropyl alcohol to yield 165.6 mg of 4-chloro-5-[4-methoxy-3-(2-methoxyethoxy)phenyl]nicotinonitrile as an off-white solid (78%).

To a stirred solution of 4-chloro-5-[4-methoxy-3-(2-methoxyethoxy)phenyl]nicotinonitrile (100 mg, 0.313 mmol), 2,4-dichloro-5-methoxyaniline (90 mg, 0.47 mmol), 2-dicyclohexylphosphino-2′-(N,N-dimethylamino)biphenyl (37 mg, 0.094 mmol), and K₃PO₄ (99.8 mg, 0.47 mmol) in 4 mL anhydrous ethylene glycol dimethyl ether was added Pd₂(dba)₃ (28.7 mg, 0.031 mmol). The mixture was heated to 90° C. for 2 hours, then cooled, filtered through Celite®, concentrated in vacuo, and purified by tritration with ether/hexane to yield 19 mg (13%) of 4-[(2,4-dichloro-5-methoxyphenyl)amino]-5-[4-methoxy-3-(2-methoxyethoxy)phenyl]nicotinonitrile 148. HPLC retention time^{(c)}: 11.99 min.; MS [M+H]: 474.1.

### EXAMPLE 10

### Preparation of 4-(2,4-dichloro-5-methoxy-phenylamino)-5-[3-methoxy-4-(2-methoxy-ethoxy)-phenyl]-nicotinonitrile 149

To a stirred solution of ethyl homovanillate (16.2 g, 77.05 mmol), tetrabutylammonium iodide (TBAI, 1.42 g, 3.85 mmol), and 2-bromoethylmethyl ether (10.4 mL, 115.5 mmol) in 250 mL of acetone was added cesium carbonate (Cs2CO3, 40.16 g, 123.2 mmol). The mixture was stirred for 21.5 hours at reflux. The mixture was concentrated and the residue was extracted from water with EtOAc. The combined organic extracts were then dried over Na2SO4, filtered, and concentrated in vacuo to yield 27 g of orange oil. The oil was purified by flash chromatography using silica gel and 10-60% EtOAc/hexane. Combined fractions were concentrated to give 20.67 g (100%) of [3-methoxy-4-(2-methoxyethoxy)phenyl]acetic acid ethyl ester as a colorless oil.

To a 500 mL three-necked round-bottomed flask was added 100 mL of anhydrous THF and cooled to −78oC. n-Butyl lithium (1.6 M in hexane, 69.8 mL, 111.8 mmol) was added to the flask and let stir for 5 minutes. Anhydrous CH₃CN (6.02 mL, 115.3 mmol) in 50 mL of anhydrous THF was added drop-wise to the flask with stirring and cooling to −78oC. After 1 hour of stirring, [3-methoxy-4-(2-methoxyethoxy)phenyl]acetic acid ethyl ester (10 g, 37.2 mmol) in 60 mL of anhydrous THF was added drop-wise to the resulting white colloidal mixture in the flask. The reaction mixture was stirred for an additional 2 hours, followed by the addition of saturated aqueous NH4Cl solution at −78oC. The solution was warmed to room temperature, diluted with 200 mL water and extracted with EtOAc (3×200 mL). The organic layer was separated, washed with brine, dried with anhydrous MgSO4, filtered, and concentrated in vacuo. The crude was purified by silica gel chromatography eluting with 20-80% EtOAc in hexanes to yield 7.39 mg (75%) of 4-[3-methoxy-4-(2-methoxyethoxy)phenyl]-3-oxo-butyronitrile as a yellow solid.

To a stirred solution of 4-[3-methoxy-4-(2-methoxyethoxy)phenyl]-3-oxo-butyronitrile (7.22 g, 27.4 mmol) in 16 mL anhydrous DMF was added DMF-DMA (16 mL, 120.6 mmol) and the solution heated at 100° C. for 15 hours. The reaction was concentrated in vacuo and then the crude material was stirred with 3,4-dimethoxybenzylamine (4.95 mL, 32.8 mmol) in 20 mL of anhydrous toluene at reflux for 2 hours. The reaction was cooled, concentrated in vacuo, and purified by silica gel chromatography eluting with 50-100% EtOAc/hexane to yield 8.26 g (67%) of 1-(3,4-dimethoxy-benzyl)-5-[3-methoxy-4-(2-methoxyethoxy)-phenyl]-4-oxo-1,4-dihydro-pyridine-3-carbonitrile as a yellow solid.

A solution of 1-(3,4-dimethoxybenzyl)-5-[3-methoxy-4-(2-methoxyethoxy)phenyl]-4-oxo-1,4-dihydro-pyridine-3-carbonitrile (8.13 g, 18 mmol) and LiCl (6.8 g, 162.4 mmol) in 65 mL POCl3 was heated at reflux for 2.5 h. The excess POCl₃ was removed by concentrating in vacuo and then the residue was co-evaporated with toluene. The residue was dissolved in 100 mL ethyl acetate and washed with ice-cold 1 N aqueous NaOH. The organic layer was separated, dried over anhydrous MgSO₄, filtered, concentrated in vacuo, and the resulting solid was triturated with isopropyl alcohol to yield 4.49 g of 4-chloro-5-[3-methoxy-4-(2-methoxyethoxy)phenyl]nicotinonitrile as an off-white solid (78%).

Following procedures analogous to those described for the preparation of compound 148 in Example 9,4-(2,4-dichloro-5-methoxyphenylamino)-5-[3-methoxy-4-(2-methoxy ethoxy)phenyl]nicotinonitrile 149 was prepared as an off-white solid, with a yield of 27 mg (18%). MS: 474.1 m/z; HPLC retention time^{(c)}: 12.0 min.

### EXAMPLE 11

### Preparation of 5-[3-(2-chloroethoxy)phenyl]-4-[(2,4-dichloro-5-methoxyphenyl)amino]nicotinonitrile 150 and 4-[(2,4-dichloro-5-methoxyphenyl)amino]-5-[3-(2-pyrrolidin-1-ylethoxy)phenyl]nicotinonitrile 151

To a stirred solution of 3-hydroxyphenylacetic acid methyl ester (22.6 g, 136 mmol) and 2-chloroethyl p-toluenesulfonate (40 g) in 0.9 L acetone was added Cs2CO3 (88.8 g) and heated at reflux for 3 hours. The mixture was then cooled, filtered, and concentrated in vacuo. The residue was purified by silica gel chromatography eluting with 0-7% EtOAc in hexanes to yield [3-(2-chloroethoxy)phenyl]acetic acid methyl ester as a colorless oil, 28.9 g (90%).

To a 1.0 L three-necked round-bottomed flask was added 150 mL of anhydrous THF and cooled to −78° C. n-Butyl lithium (2.5 M in hexane, 52.5 mL, 131 mmol) was added dropwise to the flask and its contents. Anhydrous CH3CN (7.2 mL, 138 mmol) in 150 mL of anhydrous THF was added dropwise to the flask amidst stirring and cooling. After 1 hour of stirring, 15 g [3-(2-chloroethoxy)phenyl]-acetic acid methyl ester (66 mmol) in 20 mL of anhydrous THF was added dropwise to the resulting white colloidal mixture in the flask. The reaction mixture was stirred for a further 2 hours, followed by the addition of 4:1 mixture of MeOH:AcOH at −78° C. The solution was diluted with 500 mL water and extracted with EtOAc (4×150 mL). The organic layer was separated, dried with anhydrous MgSO₄, filtered, and concentrated in vacuo. Residual AcOH was removed by concentrating in vacuo with toluene. The residue was passed through silica gel with CH₂Cl₂ to yield 4-[3-(2-chloroethoxy)phenyl]-3-oxo-butyronitrile as an off-white solid, 16 g (99%).

To a stirred solution of 4-[3-(2-chloroethoxy)phenyl]-3-oxo-butyronitrile (16 g, 67 mmol) in 100 mL anhydrous DMF was added DMF-DMA (17.6 g, 19.74 mL, 148 mmol), triethylamine (9.4 mL, 67 mmol), and the solution heated at 100° C. for 2.5 hours. The reaction was concentrated in vacuo then dissolved in CH₂Cl₂ and passed through Magnesol®. The crude material was then stirred with 3,4-dimethoxybenzylamine (11 mL, 74 mmol) in 100 mL of anhydrous toluene at reflux for 2 hours. The reaction was cooled, concentrated in vacuo, and purified by silica gel chromatography eluting with EtOAc to yield 11.8 g (41%) of 5-[3-(2-chloroethoxy)phenyl]-1-(3,4-dimethoxybenzyl)-4-oxo-1,4-dihydro-pyridine-3-carbonitrile as an off-white solid.

A solution of 5-[3-(2-chloroethoxy)phenyl]-1-(3,4-dimethoxybenzyl)-4-oxo-1,4-dihydro-pyridine-3-carbonitrile 52 (2.5 g, 5.9 mmol) and LiCl (2.3 g, 53 mmol) in 22 mL POCl3 was heated at reflux for 2.5 hours. The excess POCl₃ was removed by concentrating in vacuo. The residue was dissolved in 100 mL CH₂Cl₂ and washed with ice cold 3 N NaOH. The organic layer was separated, dried over anhydrous MgSO₄, filtered, concentrated in vacuo, and purified by silica gel chromatography eluting with 30% EtOAc in hexanes to yield 1.3 g of 4-chloro-5-[3-(2-chloroethoxy)phenyl]nicotinonitrile as an off-white solid (75%).

To a stirred solution of 4-chloro-5-[3-(2-chloroethoxy)phenyl]nicotinonitrile (200 mg, 0.68 mmol), 2,4-dichloro-5-methoxyaniline (196 mg, 1 mmol), 2-dicyclohexylphosphino 2′(N,N-dimethylamino)biphenyl (80 mg, 0.20 mmol), and K₃PO₄ (216 mg, 1 mmol) in 4 mL anhydrous ethylene glycol dimethyl ether was added Pd₂(dba)₃ (62 mg, 0.07 mmol). The mixture was heated to 90° C. for 2 hours then cooled, filtered through Celite®, concentrated in vacuo, and purified by silica gel chromatography eluting with 5-50% MeOH in CH₂Cl₂ to yield 160 mg of 5-[3-(2-chloroethoxy)phenyl]-4-[(2,4-dichloro-5-methoxyphenyl)amino]nicotinonitrile 150 as a solid (52%). HPLC retention time^{(c)}: 14.29 min.; MS: 448 [M+H].

A stirred solution of 5-[3-(2-chloroethoxy)phenyl]-4-[(2,4-dichloro-5-methoxyphenyl)amino]nicotinonitrile 150 (138 mg, 0.31 mmol), pyrrolidine (66 mg, 0.93 mmol) in 2.5 mL EtOH was heated to 105° C. for 7 hours. The reaction was cooled then poured into 25 mL of water and chilled to 0° C. The solid was filtered and dried in vacuo at 50° C. overnight to yield 32 mg of 4-[(2,4-dichloro-5-methoxyphenyl)amino]-5-[3-(2-pyrrolidin-1-ylethoxy)phenyl]nicotinonitrile 151 as a brown solid (21%). HPLC retention time^{(c)}: 6.21 min.; MS: 481 [M+H].

### EXAMPLE 12

### Preparation of 5-[4-(dimethylamino)phenyl]-4-[(3-nitrophenyl)amino]nicotinonitrile 152

4-Chloro-5-[4-(dimethylamino)phenyl]nicotinonitrile was prepared from 4-(dimethylamino)phenyl acetic acid using procedures analogous to those described for the preparation of 4-chloro-5-(3-nitrophenyl)nicotinonitrile in Example 5. The resultant 4-chloro-5-[4-(dimethylamino)phenyl]nicotinonitrile was reacted with 3-nitroaniline following procedures analogous to those described for the preparation of compound 137 in Example 5 to yield 5-[4-(dimethylamino)phenyl]-4-[(3-nitrophenyl)amino]nicotinonitrile 152.

Compound 152 was analyzed by HPLC under the following conditions: Column YMC C18, 4.6×500 mm, 5 microns; Mobile Phase A: 90% water+10% MeOH+0.02% H3PO4; Mobile Phase B: 90% MeOH+10% water+0.02% H₃PO4; 1-100% B in 2 min., up to 10 min. 100% B, then 100-1% B in 1 min. HPLC retention time^{(c)}: 3.4 min.; MS: 357.8 m/e (M−H).

Using procedures analogous to those described for the preparation of compound 147, compounds 153-158 in Table 5 were prepared starting from 3-methoxyphenyl acetic acid.

TABLE 5

| | | HPLC | |
|---|---|---|---|
| | | Retention | Observed Ion |
| | | Time | m/e |
| Compound | Compound Name | (min) | [M + H] |
| 153 | 5-(3-methoxyphenyl)-4-[(3-nitrophenyl)amino] | 3.4^{(c)} | 346.8 |
| | nicotinonitrile | | |
| 154 | 5-(3-methoxyphenyl)-4-[(3-methoxyphenyl)amino] | 3.2^{(c)} | 332.3 |
| | nicotinonitrile | | |
| 155 | 4-[(3-fluorophenyl)amino]-5-(3-methoxyphenyl) | 3.3^{(c)} | 320.1 [M − H] |
| | nicotinonitrile | | |
| 156 | 4-{[3-cyano-5-(3-methoxyphenyl)pyridin-4-yl] | 3.1^{(c)} | 346.3 |
| | amino}benzoic acid | | |
| 157 | 4-[(4-cyanophenyl)amino]-5-(3-methoxyphenyl) | 3.3^{(c)} | 327.4 |
| | nicotinonitrile | | |
| 158 | 4-[(3,4-difluorophenyl)amino]-5-(3-methoxyphenyl) | 3.4^{(c)} | 335.8 [M − H] |
| | nicotinonitrile | | |

### EXAMPLE 13

### Preparation of 5-(3,4-dimethoxyphenyl)-4-[(3-hydroxyphenyl)amino]nicotinonitrile 159

This compound was prepared by heating 4-chloro-5-(3,4-dimethoxyphenyl)nicotinonitrile with 3-aminophenol in ethanol in a sealed vial at 90° C. HPLC retention time^{(C)}: 6.4 min.; MS: 348.1 m/e (M−H).

### EXAMPLE 14

### Preparation of 5-(3,4-dimethoxyphenyl)-4-{[3-(2-hydroxyethoxy)phenyl]amino}nicotinonitrile 160

To a mixture of 5-(3,4-dimethoxyphenyl)-4-[(3-hydroxyphenyl)amino]nicotinonitrile 159 (100 mg, 0.29 mmol) and 2-bromoethanol (55 mg, 0.44 mmol) in DMF (2 mL) was added cesium carbonate (143 mg, 0.44 mmol). The resulting mixture was heated at 100° C. overnight, cooled to room temperature and purified by reverse phase HPLC (eluting with a gradient of 95% to 5% of water/acetonitrile containing 1% TFA) to give 20 mg (12%) of 5-(3,4-dimethoxyphenyl)-4-{[3-(2-hydroxyethoxy)phenyl]amino}nicotinonitrile 160 as a cream solid.

HPLC retention time^{(c)}: 6.5 min.; MS: 392.1 m/e (M+H).

### EXAMPLE 15

### Preparation of 4-[(3-{[(2S)-2-amino-3-phenylpropyl]-oxy}-phenyl)amino]-5-(3,4-dimethoxyphenyl)nicotinonitrile 161

To a mixture of 5-(3,4-dimethoxyphenyl)-4-[(3-hydroxyphenyl)amino]nicotinonitrile 159 (100 mg, 0.29 mmol) and tert-butyl (1S)-1-benzyl-2-hydroxyethylcarbamate (73 mg, 0.35 mmol), triphenylphosphine (91 mg, 0.35 mmol) in THF (1.0 mL) was added diethylazodicarboxylate (61 mg, 0.35 mmol) at room temperature The reaction mixture was stirred at room temperature overnight. Additional triphenylphosphine (91 mg, 0.35 mmol) and diethylazodicarboxylate (61 mg, 0.35 mmol) were added. After stirring at room temperature for an additional 24 hours, the resulting mixture was treated with TFA (0.4 mL) at 70° C. overnight and was purified by reverse phase HPLC (eluting with a gradient of 95% to 5% of water/acetonitrile containing 1% TFA) to give 15 mg (11%) of 4-[(3-{[(2S)-2-amino-3-phenylpropyl]-oxy}-phenyl)amino]-5-(3,4-dimethoxyphenyl)nicotinonitrile 161 as a cream solid. HPLC retention time^{(c)}: 6.8 min.; MS: 481.3 m/e (M+H).

### EXAMPLE 16

### Preparation of 4-[(2-chloro-5-hydroxyphenyl)amino]-5-(5-formyl-1-benzothien-2-yl)nicotinonitrile 162

A mixture of 3-aminobut-3-enenitrile (100 g, 1.22 mol) and conc. HCl (125 mL) in water (125 mL) was heated at 80° C. for 2 hours, cooled to room temperature and filtered to remove the solid. The filtrate was extracted with ethyl acetate and the combined extracts were dried over sodium sulfate, filtered and concentrated to give a semi-solid residue which was distilled under vacuum to give 77.4 g (76%) of acetoacetonitrile (73-77° C./3-5 mmHg).

A mixture of acetoacetonitrile (41 g, 493 mmol), t-butoxybis(dimethylamino)methane (86 g, 493 mmol) and N,N-dimethylformamide dimethyl acetal (263 mL, 1.97 mol) was heated at 100° C. overnight and evaporated to remove the volatiles. The residue was triturated with hexanes/ether (1:1) and the solids were collected by filtration and washed with hexanes/ether (1:1) and a minimum amount of ethyl acetate to give 64.3 g (67%) of 5-(dimethylamino)-2-[(dimethylamino)methylene]-3-oxopent-4-enenitrile as a light yellow solid, which was used in the next step without further purification.

A mixture of 5-(dimethylamino)-2-[(dimethylamino)methylene]-3-oxopent-4-enenitrile (64.3 g, 333 mmol) and ammonium acetate (126 g, 1.66 mol) in ethanol (1.8 L) was heated at reflux for 60 hours and concentrated to remove the solvent. The resultant semi-solid residue was diluted with ethyl acetate, filtered and washed with ethyl acetate followed by CH₂Cl₂. The filtrate was evaporated to a reduced volume. The precipitated solids were collected by filtration, washed with ethyl acetate and a minimum amount of ethanol to yield 4-hydroxynicotinonitrile. The process of evaporation and crystallization was repeated to obtain more solid 4-hydroxynicotinonitrile from the mother liquor. The combined off-white solids provided 20.9 g (53%). M.p. 234-236° C.

An alternate synthesis of 4-hydroxynicotinonitrile is reported in the literature. Broekman, F. W. et al., Recueil des Travaux Chimiques des Pays-Bas, 81: 792-6 (1962).

A mixture of 4-hydroxynicotinonitrile (45.7 g, 381 mmol), iodine (96.6 g, 381 mmol) and NaOH (19.8 g, 825 mmol) in water (600 mL) was heated at 85° C. overnight, cooled to room temperature and diluted with water. The precipitate was collected by filtration and washed with water to give 57.5 g (61%) of 4-hydroxy-5-iodonicotinonitrile as a tan solid, mp>245° C.

A mixture of 4-hydroxy-5-iodonicotinonitrile (57.5 g, 234 mmol) and POCl₃ (200 mL) was heated at 100° C. for 2 hours, cooled to room temperature and evaporated to remove excess POCl₃. The residue was cooled in an ice-water bath, adjusted to pH 8-9 with aqueous 10 N NaOH and extracted with EtOAc. The combined organics were washed with water and brine, dried over MgSO₄, filtered and concentrated. The resulting solid residue washed with a minimum amount of MeOH and CH₂Cl₂ to give 46.5 g (75%) of 4-chloro-5-iodonicotinonitrile as a tan solid, mp 120-122° C.

A mixture of 4-chloro-5-iodonicotinonitrile (2.0 g, 7.6 mmol) and 2-chloro-5-hydroxyaniline (1.09 g, 7.6 mmol) in EtOH (20 mL) was heated at 90° C. in a sealed vial overnight, poured into aqueous NaHCO₃ and filtered. The crude solid washed with water and dried to afford 3.0 g (quantitative yield) of 4-[(2-chloro-5-hydroxyphenyl)amino]-5-iodonicotinonitrile as a brown solid, which was used for the next step without further purification. MS (M+H): 372.1.

A mixture of 4-[(2-chloro-5-hydroxyphenyl)amino]-5-iodonicotinonitrile (500 mg, 1.35 mmol), 2-(4,4,5,5-tetramethyl-1,3,2-dioxaborolan-2-yl)benzo[b]thiophene-5-carbaldehyde (389 mg, 1.35 mmol) and Pd(PPh₃)₄ (78 mg, 0.070 mmol) in DME (10 mL) and NaHCO₃ (aq, 2M, 1.4 mL) was heated at 80° C. overnight, cooled to room temperature and concentrated to a reduced volume. The residue was partitioned between EtOAc and water. The combined organics were dried over Na₂SO₄, filtered, concentrated and purified by silica gel column chromatography to give 160 mg (30%) of 4-[(2-chloro-5-hydroxyphenyl)amino]-5-(5-formyl-1-benzothien-2-yl)nicotinonitrile 162 as a yellow solid, MS (M+H): 406.2; HPLC retention time^{(C)}: 11.7 min.

### EXAMPLE 17

### Preparation of 4-[(2-chloro-5-hydroxyphenyl)amino]-5-[5-(piperidin-1-ylmethyl)-1-benzothien-2-yl]nicotinonitrile 163

To a mixture of 4-[(2-chloro-5-hydroxyphenyl)amino]-5-(5-formyl-1-benzothien-2-yl)nicotinonitrile 162 (130 mg, 0.32 mmol) and piperidine (82 mg, 0.96 mmol) in THF (5.0 mL) was added AcOH (106 mg, 1.76 mmol). The resulting mixture was stirred at room temperature for one hour and sodium triacetoxyborohydride (203 mg, 0.96 mmol) was added. After stirring at room temperature overnight, the reaction mixture was concentrated and purified by silica gel column chromatography to give 105 mg (69%) of the title compound as a pale yellow solid. HPLC retention time^{(c)}: 7.8 min.; MS: 475.1 m/e (M+H).

Compound 164 in Table 6 was prepared following procedures analogous to those described for the preparation of compound 160 in Example 14. Compounds 165 and 166 were prepared by coupling intermediate 66 with the appropriate anilines then treating with 2-(4,4,5,5-tetramethyl-1,3,2-dioxaborolan-2-yl)benzo[b]thiophene-5-carbaldehyde following procedures analogous to those described for the preparation of compound 162 described in Example 16, followed by reductive amination following the procedures of compound 163, Example 17.

TABLE 6

| | | HPLC | |
|---|---|---|---|
| | | Retention | Observed Ion |
| | | Time | m/e |
| Compound | Compound Name | (min) | [M + H] |
| 164 | 4-{[2-chloro-5-(2-hydroxyethoxy)phenyl]amino}- | 7.6^{(c)} | 519.3 |
| | 5-[5-(piperidin-1-ylmethyl)-1-benzothien-2- | | |
| | yl]nicotinonitrile | | |
| 165 | 4-[(4-amino-2,3-dimethylphenyl)amino]-5-[5- | 5.4^{(c)} | 468.4 |
| | (piperidin-1-ylmethyl)-1-benzothien-2- | | |
| | yl]nicotinonitrile | | |
| 166 | 4-[(4-amino-3-methylphenyl)amino]-5-[5- | 5.4^{(c)} | 454.3 [M − H] |
| | (piperidin-1-ylmethyl)-1-benzothien-2- | | |
| | yl]nicotinonitrile | | |

### EXAMPLE 18

### Preparation of 4-chloro-5-iodo-1-oxy-nicotinonitrile

To a solution of 4-chloro-5-iodo-nicotinonitrile (529 mg, 2.0 mmol) in TFA (5 mL) was added H₂O₂ (30 wt % in H₂O, 5 mL). The reaction mixture was stirred at room temperature overnight, heated to 50° C. for 8 h, and concentrated. To the residue was added saturated aqueous NaHCO₃ (10 mL) followed by extraction with EtOAc/THF. The organic extracts were washed with water, dried over Na₂SO₄, filtered, and concentrated. The residue was purified by flash chromatography (CH₂Cl₂-THF=10:1) to give 202 mg (36%) of 4-chloro-5-iodo-1-oxy-nicotinonitrile as a pale-yellow solid.

### EXAMPLE 19

### Preparation of 4-fluoro-5-[3-methoxy-4-(2-methoxyethoxy)phenyl]nicotinonitrile

4-Chloro-5-(3,4-dimethoxyphenyl)nicotinonitrile (7.3 mmol, 2.0 g) was dissolved in 70 mL DMF and treated with CsF (14.6 mmol, 2.2 g). After heating for 2 h at 80° C., additional 7 mmol (1 g) of CsF was added and the heating was continued overnight. The suspension was evaporated onto silica gel and the product was purified by chromatography (EtOAc/Hex) to give 300 mg of 4-fluoro-5-[3-methoxy-4-(2-methoxyethoxy)phenyl]nicotinonitrile.

### EXAMPLE 20

### Preparation of 1-benzofuran-5-carbaldehyde

To a solution of 1-benzofuran-5-carbonitrile (5.0 g, 34.9 mmol) in CH₂Cl₂ under nitrogen at −15 to −20° C. was added DIBAL-H (41.9 mL, 41.9 mmol, 1 M/heptane) and the temperature was maintained below −15° C. After addition was complete, the reaction mixture was stirred at −15 to −20° C. for an additional 10 min. The reaction mixture was quenched via dropwise addition of aqueous 2N HCl. The organic layer was separated and washed with water, dried over sodium sulfate, and concentrated to give 4.0 g (78%) of 1-benzofuran-5-carbaldehyde as a yellow oil.

### EXAMPLE 21

### Preparation of dimethyl 5-(piperidin-1-ylmethyl)benzofuran-2-ylboronate

1-Benzofuran-5-carbaldehyde was treated with piperidine and sodium triacetoxyborohydride under standard reductive amination procedures to provide 1-(5-benzofuranylmethyl)piperidine. Treatment of 1-(5-benzofuranylmethyl)piperidine with butyl lithium and trimethylborate at low temperature provided dimethyl 5-(piperidin-1-ylmethyl)benzofuran-2-ylboronate. Compounds 167-169, 171, and 172 in Table 7 were provided following procedures analogous to those described in Scheme 10 by coupling with dimethyl 5-(piperidin-1-ylmethyl)benzofuran-2-ylboronate.

TABLE 7

| | | HPLC | |
|---|---|---|---|
| | | Retention | Observed Ion |
| | | Time | m/e |
| Compound | Compound Name | (min) | [M + H] |
| 167 | 4-[(2-chloro-5-methoxyphenyl)amino]-5-[5- | 1.99^{(d)} | 473.4 |
| | (piperidin-1-ylmethyl)-1-benzofuran-2- | | |
| | yl]nicotinonitrile | | |
| 168 | 4-[(2-chloro-5-methylphenyl)amino]-5-[5- | 2.02^{(d)} | 457.3 |
| | (piperidin-1-ylmethyl)-1-benzofuran-2- | | |
| | yl]nicotinonitrile | | |
| 169 | 4-[(5-hydroxy-2-phenoxyphenyl)amino]-5-[5- | 1.94^{(d)} | 517.3 |
| | (piperidin-1-ylmethyl)-1-benzofuran-2- | | |
| | yl]nicotinonitrile | | |
| 171 | 4-[(2,4-dichloro-5-hydroxyphenyl)amino]-5-[5- | 8.3^{(c)} | 493.3 |
| | (piperidin-1-ylmethyl)-1-benzofuran-2- | | |
| | yl]nicotinonitrile | | |
| 172 | 4-[(4-methoxy-2-methylphenyl)amino]-5-[5- | 7.2^{(c)} | 453.4 |
| | (piperidin-1-ylmethyl)-1-benzofuran-2- | | |
| | yl]nicotinonitrile | | |

### EXAMPLE 22

### Preparation of 4-{[3-(aminomethyl)benzyl]amino}-5-(3,4-dimethoxyphenyl)nicotinonitrile 170

A mixture of 4-chloro-5-(3,4-dimethoxyphenyl)nicotinonitrile (74 mg, 0.27 mmol), 1,3-phenylenedimethanamine (54 mg, 0.40 mmol) and triethylamine (40 mg, 0.40 mmol) in 3 mL of DMF was heated to 60° C. overnight. After cooled to the room temperature, the reaction was concentrated to dryness and the residue was dissolved in 3 mL DMSO, filtered, and purified by a preparative HPLC to give 4-{[3-(aminomethyl)benzyl]amino}-5-(3,4-dimethoxyphenyl)nicotinonitrile. HPLC retention time (d): 1.33 min.; MS: 375.2 m/e (M+H).

### EXAMPLE 23

### Pharmacological Testing

Evaluation of representative compounds of the present teachings in several standard pharmacological test procedures indicated that the compounds of the present teachings are inhibitors of PKCθ. Based on the activity shown in the standard pharmacological test procedures, the compounds of the present teachings are therefore useful as anti-inflammatory agents.

### A Radioactive Kinase Assay for Inhibition of the Active Kinase Domain (KD) of PKCθ

This assay is based on the phosphorylation of a biotinylated substrate by a kinase utilizing radiolabeled ATP (ATP γ P33). The substrate was a biotinylated peptide with a sequence of biotin-FARKGSLRQ-C(O)NH₂. The enzyme was purified recombinant active kinase domain of full length PKC theta (amino acids 362-706). The assay buffer was composed of 100 mM Hepes, pH7.5, 2 mM MgCl₂, 20 mM β-glycerophosphate and 0.008% TritonX 100. A reaction mixture of ATP, ATP γ P33 (PerkinElmer), DTT, and the enzyme was prepared in the assay buffer and added to a 96-well polypropylene plate. The compound (diluted in DMSO in a separate 96-well polypropylene plate) was added to the reaction mixture and incubated at room temperature. Following the incubation, the peptide substrate was added to the reaction mixture to initiate the enzymatic reaction. The reaction was terminated with the addition of a stop solution (100 mM EDTA, 0.2% TritonX100, and 20 mM NaHPO₄) and transferred from the assay plate to a washed streptavidin-coated 96-well scintiplate (PerkinElmer). The scintiplate was incubated at room temperature, washed in PBS with 0.1% TritonX 100, and counted in the 1450 Microbeta Trilux (Wallac, Version 2.60). Counts were recorded for each well as corrected counts per minute (CCPM). The counts were considered corrected because they were adjusted according to a P33 normalization protocol, which corrects for efficiency and background differences between the instrument detectors (software version 4.40.01).

### A Radioactive Kinase Assay for Inhibition of Full Length (FL) PKCθ Inhibitors

This assay differs from what was described above in that the enzyme used was purified recombinant full length PKC theta (Panvera, P2996).

### PKCθ IMAP Assay

The materials used include the following: human PKCθ full length enzyme (Panvera Cat# P2996); substrate peptide: 5FAM-RFARKGSLRQKNV-OH (Molecular Devices, RP7032); ATP (Sigma Cat # A2383); DTT (Pierce, 20291); 5× kinase reaction buffer (Molecular Devices, R7209); 5× binding buffer A (Molecular Devices, R7282), 5× binding buffer B (Molecular Devices, R7209); IMAP Beads (Molecular Devices, R7284); and 384-well plates (Corning Costar, 3710).

The reaction buffer was prepared by diluting the 5× stock reaction buffer and adding DTT to obtain a concentration of 3.0 mM. The binding buffer was prepared by diluting the 5× binding buffer A. A master mix solution was prepared using a 90% dilution of the reaction buffer containing 2× ATP (12 uM) and 2× peptide (200 nm). Compounds were diluted in DMSO to 20× of the maximum concentration for the IC50 measurement. 27 μl of the master mix solution for each IC50 curve was added to the first column in a 384-well plate and 3 μl of 20× compound in DMSO was added to each well. The final concentration of compound was 2× and 10% DMSO. DMSO was added to the rest of the master mix to increase the concentration to 10%. 10 μl of the master mix containing 10% DMSO was added to the rest of the wells on the plate except the 2nd column. 20 μl was transferred from the first column to the 2nd column.

The compounds were serially diluted in 2:1 ratio starting from the 2nd column. A 2× (2 nM) PKCθ solution was made in the reaction buffer. 10 μl of the PKCθ solution was added to every well to achieve these final concentrations: PKCθ—1 nM; ATP—6 μM; peptide—100 nM; DMSO—5%. Samples were incubated for 25 minutes at room temperature. The binding reagent was prepared by diluting the beads in 1× binding buffer to 800:1. 50 μl of the binding reagent was added to every well and incubated for 20 minutes. FP was measured using Envision2100 (PerkinElmer Life Sciences). Wells with no ATPs and wells with no enzymes were used as controls.

The results obtained are summarized in Table 8 below. Data presented represent the average value when one or more samples were tested.

TABLE 8

| Compound | IC₅₀ PKCθ KD | IC₅₀ PKCθ FL | IC₅₀ PKCθ IMAP |
|---|---|---|---|
| Number | (μM) | (μM) | (uM) |
| 101 | 6.87 | N/A | N/A |
| 104 | 0.60 | N/A | N/A |
| 105 | 10.60 | N/A | N/A |
| 106 | 0.84 | N/A | N/A |
| 107 | 2.91 | N/A | N/A |
| 108 | 2.77 | N/A | N/A |
| 109 | 0.81 | N/A | N/A |
| 110 | 1.16 | N/A | N/A |
| 111 | 1.41 | N/A | N/A |
| 113 | 3.60 | N/A | N/A |
| 114 | 0.40 | N/A | N/A |
| 115 | 4.26 | 0.38 | N/A |
| 116 | 3.20 | N/A | N/A |
| 117 | 13.80 | N/A | N/A |
| 118 | 4.44 | N/A | N/A |
| 119 | 11.70 | N/A | 1.95 |
| 122 | 1036 | N/A | N/A |
| 123 | 38.00 | N/A | N/A |
| 126 | 0.34 | N/A | N/A |
| 127 | N/A | N/A | 0.57 |
| 128 | N/A | N/A | 35 |
| 129 | N/A | N/A | 36 |
| 130 | N/A | N/A | 0.80 |
| 131 | N/A | N/A | 0.71 |
| 133 | N/A | N/A | 30.0 |
| 134 | N/A | N/A | 2.03 |
| 135 | 5.0 | N/A | N/A |
| 137 | 0.19 | 0.16 | 0.18 |
| 138 | N/A | 0.32 | N/A |
| 139 | N/A | 8.00 | N/A |
| 140 | N/A | 9.75 | N/A |
| 141 | N/A | 0.16 | 0.10 |
| 142 | 214 | N/A | N/A |
| 144 | 158 | N/A | N/A |
| 146 | 3.85 | N/A | N/A |
| 148 | N/A | 0.52 | N/A |
| 149 | N/A | 7.09 | N/A |
| 150 | N/A | 0.50 | N/A |
| 151 | N/A | 7.10 | N/A |
| 152 | N/A | N/A | 7.84 |
| 153 | N/A | N/A | 9.63 |
| 154 | N/A | N/A | 1.73 |
| 155 | N/A | N/A | 1.57 |
| 156 | N/A | N/A | 46.4 |
| 157 | N/A | N/A | >95 |
| 158 | N/A | N/A | 2.40 |
| 159 | N/A | N/A | 0.31 |
| 160 | N/A | N/A | 5.21 |
| 161 | N/A | N/A | 0.82 |
| 162 | N/A | N/A | 0.03 |
| 163 | N/A | N/A | 0.03 |
| 164 | N/A | N/A | 0.06 |
| 165 | N/A | N/A | 0.65 |
| 166 | N/A | N/A | 1.06 |
| 167 | N/A | N/A | 0.73 |
| 168 | N/A | N/A | 2.04 |
| 169 | N/A | N/A | 0.65 |
| 170 | N/A | N/A | 11.90 |
| 171 | N/A | N/A | 0.27 |
| 172 | N/A | N/A | 0.16 |

Variations, modifications, and other implementations of what is described herein will occur to those of ordinary skill in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced therein.

What is claimed is:

1. A compound of formula I or formula I′: 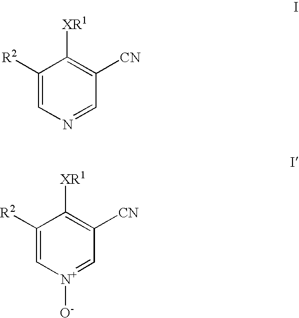
or a pharmaceutically acceptable salt, hydrate, or ester thereof, wherein:
X is selected from a) —NR³—Y—, b) —O—Y—, c) —S(O)ₘ—Y—, d) —S(O)ₘNR³—Y—, e) —NR³S(O)ₘ—Y—, f) —C(O)NR³—Y—, g) —C(S)NR³—Y—, h) —NR³C(O)—Y—, i) —NR³C(S)—Y—, j) —C(O)O—Y—, k) —OC(O)Y—, and 1) a covalent bond;
Y, at each occurrence, independently is selected from a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, and e) a covalent bond;
R¹ is a phenyl group optionally substituted with 1-4 —Y—R⁴ groups;
R² is a C₆₋₁₄ aryl group or a 5-14 membered heteroaryl group, wherein each group optionally is substituted with 1-4 groups independently selected from —Y—R⁴ or —O—Y—R⁴;
R³ is selected from a) H, b) a C₁₋₁₀ alkyl group, c) a C₂₋₁₀ alkenyl group, d) a C₂₋₁₀ alkynyl group, and e) a C₁₋₁₀ haloalkyl group;
R⁴, at each occurrence, independently is selected from a) halogen, b) —CN, c) —NO₂,
d) oxo, e) —O—Y—R⁵, f) —NR⁶—Y—R⁷, g) —N(O)R⁶—Y—R⁷, h) —S(O)ₘ—Y—R⁵, i) —S(O)ₘO—Y—R⁵, j) —S(O)ₘNR⁶—Y—R⁷, k) —C(O)—Y—R⁵, l) —C(O)O—Y—R⁵, m) —C(O)NR⁶—Y—R⁷, n) —C(S)NR⁶—Y—R⁷, o) a C₁₀ alkyl group, p) a C₂₋₁₀ alkenyl group, q) a C₂₋₁₀ alkynyl group, r) a C₁₋₁₀ haloalkyl group, s) a C₃₋₁₄ cycloalkyl group, t) a C₆₋₁₄ aryl group, u) a 3-14 membered cycloheteroalkyl group, and v) a 5-14 membered heteroaryl group, wherein each of o)-v) optionally is substituted with 1-4 —Y—R⁸ groups;
R⁵, at each occurrence, independently is selected from a) H, b) —C(O)R⁹, c) —C(O)OR⁹, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₄ cycloalkyl group, i) a C₆₋₁₄ aryl group, j) a 3-14 membered cycloheteroalkyl group, and k) a 5-14 membered heteroaryl group, wherein each of d)-k) optionally is substituted with 1-4 —Y—R⁸ groups;
R⁶ and R⁷, at each occurrence, independently are selected from a) H, b) —O—Y—R⁹, c) —S(O)ₘ—Y—R⁹, d) —S(O)ₘO—Y—R⁹, e) —C(O)Y—R⁹, f) —C(O)O—Y—R⁹, g) —C(O)NR¹⁰—Y—R¹¹, h) —C(S)NR¹⁰—Y—R¹¹, i) a C₁₋₁₀ alkyl group, j) a C₂₋₁₀ alkenyl group, k) a C₂₋₁₀ alkynyl group, 1) a C₁₋₁₀ haloalkyl group, m) a C₃₋₁₄ cycloalkyl group, n) a C₆₋₁₄ aryl group, o) a 3-14 membered cycloheteroalkyl group, and p) a 5-14 membered heteroaryl group, wherein each of i)-p) optionally is substituted with 1-4 —Y—R⁸ groups;
R⁸, at each occurrence, independently is selected from a) halogen, b) —CN, c) —NO₂, d) oxo, e) —O—Y—R⁹, f) —NR¹⁰—Y—R¹, g) —N(O)R¹⁰—Y—R¹¹, h) —S(O)ₘ—Y—R⁹, i) —S(O)ₘO—Y—R⁹, j) —S(O)ₘNR¹⁰—Y—R¹¹, k) —C(O)—Y—R⁹, l) —C(O)O—Y—R⁹, m) —C(O)NR¹⁰—Y—R¹¹, n) —C(S)NR¹⁰Y—R¹¹, o) a C₁₋₁₀ alkyl group, p) a C₂₋₁₀ alkenyl group, q) a C₂₋₁₀ alkynyl group, r) a C₁₋₁₀ haloalkyl group, s) a C₃₋₁₄ cycloalkyl group, t) a C₆₋₁₄ aryl group, u) a 3-14 membered cycloheteroalkyl group, and v) a 5-14 membered heteroaryl group, wherein each of o)-v) optionally is substituted with 1-4 —Y—R¹² groups;
R⁹, at each occurrence, independently is selected from a) H, b) —C(O)—C₁₋₁₀ alkyl, c) —C(O)OH, d) —C(O)O—C₁₋₁₀ alkyl, e) a C₁₋₁₀ alkyl group, f) a C₂₋₁₀ alkenyl group, g) a C₂₋₁₀ alkynyl group, h) a C₁₋₁₀ haloalkyl group, i) a C₃₋₁₄ cycloalkyl group, j) a C₆₋₁₄ aryl group, k) a 3-14 membered cycloheteroalkyl group, and 1) a 5-14 membered heteroaryl group, wherein each of the C₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Y—R¹² groups;
R¹⁰ and R¹¹, at each occurrence, independently are selected from a) H, b) —OH, c) —SH, d) —NH₂, e) —NH—C₁₋₁₀ alkyl, f) —N(C₁₋₁₀ alkyl)₂, g) —S(O)ₘ-C₁₋₁₀ alkyl, h) —S(O)₂OH, i) —S(O)ₘ—OC₁₋₁₀ alkyl, j) —C(O)—₁₋₁₀ alkyl, k) —C(O)OH, 1)—C(O)OC₁₋₁₀ alkyl, m) —C(O)NH₂, n) —C(O)NH-C₁₋₁₀ alkyl, o) —C(O)N(C₁₋₁₀ alkyl)₂, p) —C(S)NH₂, q) —C(S)NH—C₁₋₁₀ alkyl, r) —C(S)N(C₁₋₁₀ alkyl)₂, s) a C₁₋₁₀ alkyl group, t) a C₂₋₁₀ alkenyl group, u) a C₂₋₁₀ alkynyl group, v) a C₁₋₁₀ alkoxy group, w) a C₁₋₁₀ haloalkyl group, x) a C₃₋₁₄ cycloalkyl group, y) a C₆₋₁₄ aryl group, z) a 3-14 membered cycloheteroalkyl group, and aa) a 5-14 membered heteroaryl group, wherein each of the C₁₋₁₀ alkyl group, the C₂₋₁₀ alkenyl group, the C₂₋₁₀ alkynyl group, the C₁₋₁₀ alkoxy group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Y—R¹² groups;
R¹², at each occurrence, independently is selected from a) halogen, b) —CN, c) —NO₂, d) oxo, e) —OH, f) —NH₂, g) —NH(C₁₋₁₀ alkyl), h) —N(C₁₋₁₀alkyl)₂, i) —SH, j) —S(O)ₘ—C₁₋₁₀alkyl, k) —S(O)₂OH, l) —S(O)ₘ—OC₁₋₁₀ alkyl, m) —C(O)—C₁₋₁₀alkyl, n) —C(O)OH, o) —C(O)—OC₁₋₁₀alkyl, p) —C(O)NH₂, q) —C(O)NH—C₁₋₁₀alkyl, r) —C(O)N(C₁₋₁₀ alkyl)₂, s) —C(S)NH₂, t) —C(S)NH—C₁₋₁₀ alkyl, u) —C(S)N(C₁₋₁₀ alkyl)₂, v) a C₁₋₁₀ alkyl group, w) a C₂₋₁₀ alkenyl group, x) a C₂₋₁₀ alkynyl group, y) a C₁₋₁₀ alkoxy group, z) a C₁₋₁₀ haloalkyl group, aa) a C₃₋₁₄ cycloalkyl group, ab) a C₆₋₁₄ aryl group, ac) a 3-14 membered cycloheteroalkyl group, and ad) a 5-14 membered heteroaryl group; and
m is 0, 1, or 2;
provided that when R¹ is a 3-chloro-4-fluorophenyl group, R² is not a 2-[(1H-imidazol-5-ylmethyl)amino]phenyl group.

2. The compound of claim 1 or a pharmaceutically acceptable salt, hydrate, or ester thereof, wherein X is selected from —NH—, —N(CH₃), —NH—CH₂—, —NH—CH₂CH₂—, —NH—CH₂CH₂CH₂—, —O—, and a covalent bond.

3. The compound of claim 1 or a pharmaceutically acceptable salt, hydrate, or ester thereof, wherein R¹ is selected from:

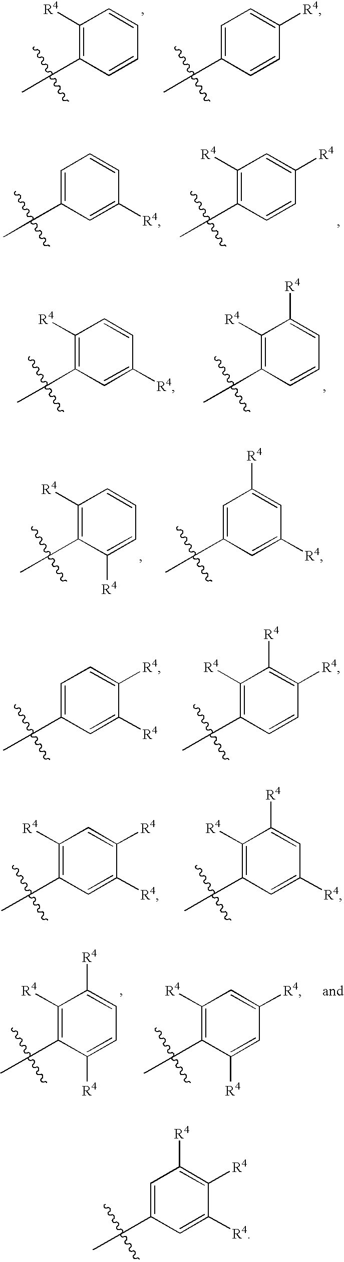

4. The compound of claim 1 or a pharmaceutically acceptable salt, hydrate, or ester thereof, wherein R⁴, at each occurrence, is independently selected from —F, —Cl, —Br, —CN, —NO₂, —O—Y—R⁵, —C(O)—Y—R⁵, —C(O)O—Y—R⁵, —NR⁶—Y—R⁷, and a C₁₋₆ alkyl group.

5. The compound of claim 1 or a pharmaceutically acceptable salt, hydrate, or ester thereof, wherein R² is selected from a phenyl group, a C₈₋₁₄ aryl group and a 5-14 membered heteroaryl group, wherein each group optionally is substituted with 1-4 groups independently selected from —Y—R⁴ and —O—Y—R⁴.

6. The compound of claim 1 or a pharmaceutically acceptable salt, hydrate, or ester thereof, wherein R² is: 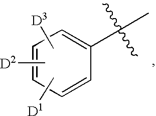
wherein D¹, D², and D³ independently are H, a —Y—R⁴ group, or an —O—Y—R⁴ group.

7. The compound claim 6 or a pharmaceutically acceptable salt, hydrate, or ester thereof, wherein at least one of D¹, D², and D³ is a —Y—R⁴ group or an —O—Y—R⁴ group, wherein Y, at each occurrence, independently is a divalent C₁₋₄ alkyl group or a covalent bond, and R⁴, at each occurrence, independently is selected from a halogen, —CN, NO₂, —O—Y—R⁵, —NR⁶—Y—R⁷, —S(O)₂—Y—R⁵, —S(O)₂NR⁶—Y—R⁷, —C(O)Y—R⁵, —C(O)O—Y—R⁵, —C(O)NR⁶—Y—R⁷, a C₁₋₁₀ alkyl group, a C₁₋₁₀ haloalkyl group, a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group is optionally substituted with 1-4 —Y—R⁸ groups.

8. The compound of claim 7 or a pharmaceutically acceptable salt, hydrate, or ester thereof, wherein the —Y—R⁴ group and the —O—Y—R⁴ group are selected from —O—(CH₂)ₙNR⁶—Y—R⁷, —CH₂)ₙNR⁶—Y—R⁷, an —O—(CH₂)ₙ-3-14 membered cycloheteroalkyl group, and a —(CH₂)ₙ-3-14 membered cycloheteroalkyl group, wherein each of the 3-14 membered cycloheteroalkyl group optionally is substituted with 1-4-Y—R⁸ groups, and n, at each occurrence, independently is 0, 1, 2, 3, or 4.

9. The compound of claim 8 or a pharmaceutically acceptable salt, hydrate, or ester thereof, wherein the 3-14 membered cycloheteroalkyl group of the —O—(CH₂)ₙ-3-14 membered cycloheteroalkyl group and the —(CH₂)ₙ-3-14 membered cycloheteroalkyl group is selected from a pyrrolidinyl group, a morpholinyl group, a piperazinyl group, a piperidinyl group, an azepanyl group, a diazepanyl group, and a thiomorpholinyl group.

10. The compound of claim 7 or a pharmaceutically acceptable salt, hydrate, or ester thereof, wherein the —Y—R⁴ group and the —O—Y—R⁴ group are 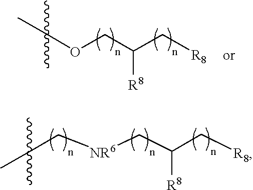
wherein R⁸, at each occurrence, independently is selected from —O—Y—R⁹, —NR¹⁰—Y—R¹¹, a C₆₋₁₄ aryl group, and a 5-14 membered heteroaryl group, wherein the C₆₋₁₄ aryl group and the 5-14 membered heteroaryl group optionally are substituted with 1-4 —Y—R¹² groups, and n, at each occurrence, independently is 0, 1, 2, 3, or 4.

11. The compound of claim 7 or a pharmaceutically acceptable salt, hydrate, or ester thereof, wherein at least one of D¹, D², and D³ is selected from a halogen, —CN, —NO₂, —S(O)₂—Y—R⁵, —S(O)₂NR⁶—Y—R⁷, —C(O)O—Y—R⁵, —C(O)NR⁶—Y—R⁷, a C₁₋₁₀ alkyl group, and a C₁₋₁₀ haloalkyl group.

12. The compound of claim 7 or a pharmaceutically acceptable salt, hydrate, or ester thereof, wherein at least one of D¹, D², and D³ is a C₆₋₁₄ aryl group or a 5-14 membered heteroaryl group, wherein each group optionally is substituted with 1-4 —Y—R⁸ groups.

13. The compound of claim 12 or a pharmaceutically acceptable salt, hydrate, or ester thereof, wherein at least one of D¹, D², and D³ is selected from a benzothienyl group, a benzofuryl group, a furyl group, a pyridyl group, a pyrimidinyl group, a pyrrolyl group, and a thienyl group, wherein each group optionally is substituted with 1-4 —Y—R⁸ groups, Y, at each occurrence, is independently a C₁₋₄ alkyl group or a covalent bond, and R⁸, at each occurrence, is independently selected from halogen, —CN, —NO₂, —O—Y—R⁹, —NR¹⁰—Y—R¹¹, —C(O)—Y—R⁹, —C(O)NR¹⁰—Y—R¹, —S(O)₂—Y—R⁹, —S(O)₂NR¹⁰—Y—R¹¹, and a 3-14 membered cycloheteroalkyl group optionally substituted with a C₁₋₄ alkyl group.

14. The compound of claim 1 or a pharmaceutically acceptable salt, hydrate, or ester thereof, wherein R² is a C₈₋₁₄ bicyclic aryl group or a 5-14 membered heteroaryl group, wherein each of the C₈₋₁₄ bicyclic aryl group and the 5-14 membered heteroaryl group is optionally substituted with 1-4 groups independently selected from —Y—R⁴ and —O—Y—R⁴.

15. The compound of claim 14 or a pharmaceutically acceptable salt, hydrate, or ester thereof, wherein R² is selected from a pyridyl group, a pyrimidyl group, a pyrazinyl group, a furyl group, a thienyl group, a thiazolyl group, an oxazolyl group, a benzofuranyl group, a benzothienyl group, an indolyl group, a benzodioxinyl group, a benzodioxolyl group, a benzodioxanyl group, a dibenzofuranyl group, a dibenzothienyl group, a benzoindolyl group, an indanyl group, an indenyl group, an isothiazolyl group, a pyridazinyl group, a pyrazolyl group, a tetrahydronaphthyl group, an isoxazolyl group, a quinolinyl group, a naphthyl group, an imidazolyl group, and a pyrrolyl group, wherein each group optionally is substituted with 1-4 groups independently selected from —(CH₂)ₙ—R⁴ and —O—(CH₂)ₙ—R⁴, wherein n, at each occurrence, independently is 0, 1, 2, 3, or 4, and R⁴, at each occurrence, independently is —NR⁶—Y—R⁷ or a 3-14 membered cycloheteroalkyl group optionally substituted with a —Y—R⁸ group.

16. A compound of claim 1 selected from the following compounds:
4-[(3-chlorophenyl)amino]-5-(3,4-dimethoxyphenyl)nicotinonitrile,
5-(3,4-dimethoxyphenyl)-4-[(3-fluoro phenyl)amino]nicotinonitrile,
4-anilino-5-(3,4-dimethoxyphenyl)nicotinonitrile,
4-[(2,5-difluorophenyl)amino]-5-(3,4-dimethoxyphenyl)nicotinonitrile,
5-(3,4-dimethoxyphenyl)-4-[(3,4-dimethoxyphenyl)amino]nicotinonitrile,
4-[(4-chloro-2-fluorophenyl)amino]-5-(3,4-dimethoxyphenyl)nicotinonitrile,
4-[(3-chloro-4-fluorophenyl)amino]-5-(3,4-dimethoxyphenyl)nicotinonitrile,
4-[(4-chlorophenyl)amino]-5-(3,4-dimethoxyphenyl)nicotinonitrile,
5-(3,4-dimethoxyphenyl)-4-[(2,4-dimethylphenyl)amino]nicotinonitrile,
5-(3,4-dimethoxyphenyl)-4-[(4-methoxyphenyl)amino]nicotinonitrile,
4-[(3-chloro-4-methoxyphenyl)amino]-5-(3,4-dimethoxyphenyl)nicotinonitrile,
5-(3,4-dimethoxyphenyl)-4-[(4-phenoxy phenyl)amino]nicotinonitrile,
4-[(2,5-dichlorophenyl)amino]-5-(3,4-dimethoxyphenyl)nicotinonitrile,
5-(3,4-dimethoxyphenyl)-4-[(4-methoxy-2-methylphenyl)amino]nicotinonitrile,
4-[(3,4-dichlorophenyl)amino]-5-(3,4-dimethoxyphenyl)nicotinonitrile,
4-[(5-chloro-2-methoxyphenyl)amino]-5-(3,4-dimethoxyphenyl)nicotinonitrile,
4-{[3-(benzyloxy)phenyl]amino}-5-(3,4-dimethoxyphenyl)nicotinonitrile,
5-(3,4-dimethoxyphenyl)-4-[(4-methyl phenyl)amino]nicotinonitrile,
5-(3,4-dimethoxyphenyl)-4-[(3,4,5-trimethoxyphenyl)amino]nicotinonitrile,
5-(3,4-dimethoxyphenyl)-4-[(3-phenoxy phenyl)amino]nicotinonitrile,
4-[(2-chloro-5-methoxyphenyl)amino]-5-(3,4-dimethoxyphenyl)nicotinonitrile,
4-({3-chloro-4-[(3-cyanobenzyl)oxy]phenyl}amino)-5-(3,4-dimethoxy phenyl)nicotinonitrile,
4-({3-chloro-4-[(3-methylbenzyl)oxy]phenyl}amino)-5-(3,4-dimethoxyphenyl)nicotinonitrile,
4-[(3-chloro-4-{[3-(dimethylamino)benzyl]oxy}phenyl)amino]-5-(3,4-dimethoxy phenyl)nicotinonitrile,
4-[(2,4-dichlorophenyl)amino]-5-(3,4-dimethoxyphenyl)nicotinonitrile,
N-(3-{[3-cyano-5-(3,4-dimethoxyphenyl)pyridin-4-yl]amino}phenyl)acetamide,
N-(3-{[3-cyano-5-(3,4-dimethoxyphenyl)pyridin-4-yl]amino}phenyl)-N-methylacetamide,
N-(3-{[3-cyano-5-(3,4-dimethoxyphenyl)pyridin-4-yl]amino}phenyl)methanesulfonamide,
5-[4-(dimethylamino)phenyl]-4-[(3-methoxyphenyl)amino]nicotinonitrile,
5-[4-(dimethylamino)phenyl]-4-[(3-fluorophenyl)amino]nicotinonitrile,
4-({3-cyano-5-[4-(dimethylamino)phenyl]pyridin-4-yl} amino)benzoic acid,
4-[(4-cyanophenyl)amino]-5-[4-(dimethylamino)phenyl]nicotinonitrile,
4-[(3,4-difluorophenyl)amino]-5-[4-(dimethylamino)phenyl]nicotinonitrile,
4-[(3-bromophenyl)amino]-5-(3,4-dimethoxyphenyl)nicotinonitrile,
4-{[3-(benzyloxy)-4-chloro phenyl]amino}-5-(3,4-dimethoxyphenyl)nicotinonitrile,
4-[(2,4-dichloro-5-methoxyphenyl)amino]-5-(3,4-dimethoxyphenyl)nicotinonitrile,
4-[(2,4-dichloro-5-ethoxyphenyl)amino]-5-(3,4-dimethoxyphenyl)nicotinonitrile,
4-[(2,4-dichloro-5-propoxyphenyl)amino]-5-(3,4-dimethoxyphenyl)nicotinonitrile,
4-[(5-butoxy-2,4-dichlorophenyl)amino]-5-(3,4-dimethoxyphenyl)nicotinonitrile,
4-{[2,4-dichloro-5-(2-hydroxy ethoxy)phenyl]amino}-5-(3,4-dimethoxyphenyl)nicotinonitrile,
4-{[4-(benzyloxy)-3-chlorophenyl]amino}-5-(3-nitro phenyl)nicotinonitrile,
4-{[3-chloro-4-(pyridin-2-ylmethoxy)phenyl]amino}-5-(3-nitrophenyl)nicotinonitrile,
4-[(3-chloro-4-fluorophenyl)amino]-5-(3-nitrophenyl)nicotinonitrile,
5-(3-aminophenyl)-4-{[4-(benzyloxy)-3-chlorophenyl]amino}nicotinonitrile,
4-[(3-chloro-4-fluorophenyl)amino]-5-(2-nitrophenyl)nicotinonitrile,
5-(2-aminophenyl)-4-[(3-chloro-4-fluorophenyl)amino]nicotinonitrile,
4-[(2,4-dichloro-5-methoxy phenyl)amino]-5-[4-methoxy-3-(2-methoxy ethoxy)phenyl]nicotinonitrile,
4-[(2,4-dichloro-5-methoxy phenyl)amino]-5-[3-methoxy-4-(2-methoxyethoxy)phenyl]nicotinonitrile,
5-[3-(2-chloroethoxy)phenyl]-4-[(2,4-dichloro-5-methoxyphenyl)amino]nicotinonitrile,
4-[(2,4-dichloro-5-methoxyphenyl)amino]-5-[3-(2-pyrrolidin-1-ylethoxy)phenyl]nicotinonitrile,
5-[4-(dimethylamino)phenyl]-4-[(3-nitrophenyl)amino]nicotinonitrile,
5-(3-methoxyphenyl)-4-[(3-nitrophenyl)amino]nicotinonitrile,
5-(3-methoxyphenyl)-4-[(3-methoxy phenyl)amino]nicotinonitrile,
4-[(3-fluorophenyl)amino]-5-(3-methoxyphenyl)nicotinonitrile,
4-{[3-cyano-5-(3-methoxyphenyl)pyridin-4-yl]amino} benzoic acid,
4-[(4-cyanophenyl)amino]-5-(3-methoxyphenyl)nicotinonitrile,
4-[(3,4-difluorophenyl)amino]-5-(3-methoxyphenyl)nicotinonitrile,
5-(3,4-dimethoxyphenyl)-4-[(3-hydroxy phenyl)amino]nicotinonitrile,
5-(3,4-dimethoxyphenyl)-4-{[3-(2-hydroxyethoxy)phenyl]amino}nicotinonitrile,
4-[(3-{[(2S)-2-amino-3-phenyl propyl]-oxy}-phenyl)amino]-5-(3,4-dimethoxy phenyl)nicotinonitrile,
4-[(2-chloro-5-hydroxyphenyl)amino]-5-(5-formyl-1-benzo thien-2-yl)nicotinonitrile,
4-[(2-chloro-5-hydroxy phenyl)amino]-5-[5-(piperidin-1-ylmethyl)-1-benzothien-2-yl]nicotinonitrile,
4-{[2-chloro-5-(2-hydroxyethoxy)phenyl]amino}-5-[5-(piperidin-1-ylmethyl)-1-benzothien-2-yl]nicotinonitrile,
4-[(4-amino-2,3-dimethylphenyl)amino]-5-[5-(piperidin-1-ylmethyl)-1-benzothien-2-yl]nicotinonitrile,
4-[(4-amino-3-methylphenyl)amino]-5-[5-(piperidin-1-ylmethyl)-1-benzothien-2-yl]nicotinonitrile,
4-[(2-chloro-5-methoxyphenyl)amino]-5-[5-(piperidin-1-ylmethyl)-1-benzofuran-2-yl]nicotinonitrile,
4-[(2-chloro-5-methylphenyl)amino]-5-[5-(piperidin-1-ylmethyl)-1-benzofuran-2-yl]nicotinonitrile,
4-[(5-hydroxy-2-phenoxyphenyl)amino]-5-[5-(piperidin-1-ylmethyl)-1-benzofuran-2-yl]nicotinonitrile,
4-{[3-(aminomethyl)benzyl]amino}-5-(3,4-dimethoxyphenyl)nicotinonitrile,
4-[(2,4-dichloro-5-hydroxyphenyl)amino]-5-[5-(piperidin-1-ylmethyl)-1-benzofuran-2-yl]nicotinonitrile, and
4-[(4-methoxy-2-methylphenyl)amino]-5-[5-(piperidin-1-ylmethyl)-1-benzofuran-2-yl]nicotinonitrile.

17. The compound of claim 1 or a pharmaceutically acceptable salt, hydrate, or ester thereof, wherein the compound is in the form of an enantiomer.

18. A pharmaceutical composition comprising the compound of claim 1 and a pharmaceutically acceptable carrier or excipient.

19. A method of treating or inhibiting a pathological condition or disorder mediated by a protein kinase in a mammal, the method comprising providing to the mammal an effective amount of the compound of claim 1 or a pharmaceutically acceptable salt, hydrate, or ester thereof.

20. The method of claim 19, wherein the protein kinase is protein kinase C.

21. The method of claim 19, wherein the pathological condition or disorder is an inflammatory disease or an autoimmune disease selected from asthma, colitis, multiple sclerosis, psoriasis, arthritis, rheumatoid arthritis, osteoarthritis, and joint inflammation.
